SEQUENCE LISTING (6 sequences)

SEQ ID NO: 1 - DNA, 21 bp, Homo sapiens
  source 1..21: mol_type = other DNA
cctcgcatgctgataacaatt

SEQ ID NO: 2 - DNA, 21 bp, Homo sapiens
  source 1..21: mol_type = other DNA
ccacgcttcaatgagaacaac

SEQ ID NO: 3 - DNA, 21 bp, Homo sapiens
  source 1..21: mol_type = other DNA
ggaatttctggtgacatagac

SEQ ID NO: 4 - DNA, 19 bp, Homo sapiens
  source 1..19: mol_type = other DNA
gcttcgcgccgtagtctta

SEQ ID NO: 5 - protein, 264 aa, Homo sapiens
MHSKTPCGCFKPWKMADNFSLHDALSGSGNPNPQGWPGAWGNQPAGAGGYPGASYPGAYPGQAPPGAYPGQAPPGAYPGAPGAYPGAPAPGVYPGPPSGPGAYPSSGQPSATGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 6 - DNA, 958 bp, Homo sapiens
  source 1..958: mol_type = genomic DNA
gcccgcagcacctcctcgccagcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgccaccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaaaagaatctaaaccttacatgtgtaaaggtttcatgttcactgtgagtgaaaatttttacattcatcaatatccctcttgtaagtcatctacttaataaatattacagtgaattacctgtctcaa

(19) United States
(12) Patent Application Publication
(10) Pub. No.: US 2025/0228968 A1
(43) Pub. Date: Jul. 17, 2025

(54) TUMOR-INTRINSIC GALECTIN-3 SUPPRESSES MELANOMA METASTASIS

(71) Applicants: CHARLES J. DIMITROFF, CORAL GABLES, FL (US); NORHAN B. B. MOHAMMED, MIAMI, FL (US)

(72) Inventors: CHARLES J. DIMITROFF, CORAL GABLES, FL (US); NORHAN B. B. MOHAMMED, MIAMI, FL (US)

(73) Assignee: THE FLORIDA INTERNATIONAL UNIVERSITY BOARD OF TRUSTEES, MIAMI, FL (US)

(21) Appl. No.: 18/414,857

(22) Filed: Jan. 17, 2024

(51) Int. Cl. A61K 48/00; A61K 38/17; A61K 45/06; A61P 35/04; C12N 15/85

(52) U.S. Cl. CPC A61K 48/005; A61K 38/1732; A61K 45/06; A61P 35/04; C12N 15/85

(57) ABSTRACT

The present invention provides methods and composition for diagnosis, prognosis, prevention and/or treatment of cancers such as melanomas. The subject invention provides biomarkers (e.g., galectin-3 (Gal-3)) and methods for assessing the severity of a cancer/tumor and for monitoring the progressing of a cancer/tumor. The compositions according to the subject invention regulate and alter melanoma signaling, growth, and survival. The subject invention also provides molecular mechanisms governing melanoma metastasis to identify candidate diagnostic or prognostic markers and develop novel therapeutic opportunities. Further, the subject invention identifies the tumor-suppressive function of Gal-3 in melanoma cells, emphasizing the negative crosstalk between Gal-3 and NFAT1 and its role in dictating melanoma metastasis.

### GOVERNMENT SUPPORT

This invention was made with government support under CA225644 awarded by The National Institutes of Health (NIH). The government has certain rights in the invention.

### SEQUENCE LISTING

The Sequence Listing for this application is labeled “SeqList-16Jan24.xml,” which was created on Jan. 16, 2024, and is 7,134 bytes. The Sequence Listing is incorporated herein by reference in its entirety.

### BACKGROUND

Melanoma is a life-threatening melanocyte-derived skin neoplasm known for its aggressive clinical behavior and poor outcomes in patients with metastatic disease. Melanoma incidence has been increasing worldwide over the last few decades. Despite the recent breakthroughs in immunotherapy, e.g., using immune checkpoint inhibitors (ICI), and targeted therapy that have noticeably increased the survival of melanoma patients, the prognosis of patients with late-stage metastatic disease remains dismal with a 5-year survival rate of only 32%. Tumor cell resistance to these novel therapies is still a major concern. Therefore, surveillance for distant metastasis in patients with primary melanoma is fundamental to provide an accurate prognosis, an optimal treatment choice, and a personized follow-up maintenance plan.

The histopathological features of primary lesions (including Breslow thickness), sentinel lymph node biopsy, imaging studies, and routine blood testing (LDH, S100 protein) are collectively used to identify metastatic disease. However, there is no absolute predictor of metastasis in patients with primary melanoma and lifelong follow-up is crucial for early detection of potential metastasis. In-depth understanding of the molecular mechanisms that govern melanoma metastasis could uncover new diagnostic markers and help identify targets to develop innovative therapeutic strategies.

Tumor metastasis is a complex, multi-step process, in which cancer cells with certain genotypic and phenotypic alterations dislodge from the primary tumor and migrate through the bloodstream or the lymphatic system as single cells or small clusters of cells, a small fraction of which can eventually settle in distant organs, grow, and form secondary tumors. The intrinsic properties of primary tumor cells, such as their ability to invade surrounding tissues, elude immune-mediated clearance, and adapt to local tissue microenvironmental factors, are among the major determinants of their metastasis-initiating capacity. Throughout this functional cascade, several factors, including adhesion molecules, proteases, and cytokines, are either upregulated or downregulated by cancer cells to facilitate migration, invasion, and metastasis formation.

Galectins, a 15-member family of β-galactoside-binding lectins, are known as crucial regulators of several cellular events. The role of galectins in regulating tumor initiation, progression and drug resistance are now gaining attention in the cancer research field. Intracellular function of galectins ranges from pre-mRNA splicing, and pro- and anti-apoptosis via protein-protein interactions and regulation of autophagy. They lack a canonical signal secretion sequence and are routed to the extracellular surface in non-classical secretion pathways. In the extracellular milieu, galectin (Gal)-1, -3 and -9, are known to bind cell surface glycans and alter cellular signaling activities that control proliferation, death, migration, and other effector functions depending on a given cell type.

Galectin-3 (Gal-3) is the only chimera type galectin that has a single carbohydrate recognition domain (CRD) linked to a matrix metalloproteinase cleavable N-terminal domain. This characteristic structure allows oligomerization of Gal-3 monomers into pentamers through their N-terminal domains with formation of a lattice-like structure. While the carbohydrate-binding activity of pentameric Gal-3 predominates extracellularly to crosslink glycosylated proteins on cell surfaces and extracellular matrix (ECM), intracellular Gal-3 can engage in carbohydrate-independent interactions with other cytosolic proteins to impact many biological processes through the modulation of signaling pathways. Gal-3 has been found to be expressed in many cancer types, including melanoma. Membrane-bound and extracellular Gal-3 are theorized to bind cancer cell surface glycoconjugates to promote homotypic cell adhesion that facilitates the formation of apoptosis-resistant circulating emboli. Gal-3 can also interact with circulating tumor cells (CTCs) and endothelial cells (ECs) to facilitate cancer cell extravasation and organ colonization.

Gal-3 has emerged as a pleotropic promoter of cancer initiation and progression, having various cellular activities depending on the cancer context and presence of ligand-binding partners. Extracellular Gal-3, in many cases, has been shown to coordinate melanoma cell activities within the tumor microenvironment (TME) through Gal-3—ligand interactions and promote migration and survival. However, roles of intracellular Gal-3 in melanoma aggressive behavior remains ill-defined.

Melanoma poses a poor prognosis with high mortality rates upon metastasis. Exploring molecular mechanisms governing melanoma progression paves the way for developing novel approaches to control melanoma metastasis and ultimately enhance patient survival rates. Therefore, there is a need to explore the molecular basis of melanoma metastasis to identify candidate diagnostic or prognostic markers to help guide treatment decisions, and to develop novel therapeutic targets to augment current treatment strategies for metastatic melanoma.

### BRIEF SUMMARY

The present invention provides methods and composition for diagnosis, prognosis, prevention and/or treatment of cancers such as melanomas, in particular metastatic melanoma. The subject invention provides biomarkers and methods for assessing the severity of a cancer/tumor and for monitoring the progressing of a cancer/tumor. The compositions according to the subject invention regulate malignancy-associated pathways and alter melanoma signaling, growth, and survival.

In one embodiment, the subject invention identifies biomarkers that are involved in cancer metastasis. In a specific embodiment, the subject invention identifies Gal-3 as a metastasis-suppressive molecule in melanoma. In one embodiment, the subject invention identifies the tumor-suppressive function of Gal-3 in melanoma cells and, importantly, implicates intracellular Gal-3 as a prognostic indicator of metastatic risk in melanoma patients.

Advantageously, the subject invention helps meet the demand of the lack of biomarkers of melanoma metastasis and offers an immunohistopathological marker to assay, whose presence (i.e., expression) in melanoma cells is indicative of a non-metastatic state and its absence is indicative of high metastatic potential and triggers more aggressive treatment approaches.

In one embodiment, the subject invention provides methods for monitoring the metastatic behavior of melanoma. The methods of the subject invention use Gal-3 as a biomarker for diagnosing a cancer and/or assessing cancer metastasis, and for negatively regulating pro-metastasis factors, e.g., NFAT1 as well as metastasis-associated proteins.

In one embodiment, the subject invention provides a method for treating melanoma, preferably, metastatic melanoma, in a subject, the method comprising administering to the subject in need of such treatment a pharmaceutical composition comprising 1) a nucleic acid sequence that encodes Gal-3 or a nucleic acid sequence sharing at least 95% identity with the nucleic acid sequence that encodes Gal-3, and/or 2) a vector comprising a nucleic acid sequence that encodes Gal-3 or a nucleic acid sequence sharing at least 95% identity with the nucleic acid sequence that encodes Gal-3.

In one embodiment, the subject invention provides a method for treating melanoma, preferably, metastatic melanoma, in a subject, the method comprising administering to the subject in need of such treatment a pharmaceutical effective amount of 1) a nucleic acid sequence that encodes Gal-3 or a nucleic acid sequence sharing at least 95% identity with the nucleic acid sequence that encodes Gal-3, and/or 2) a vector comprising a nucleic acid sequence that encodes Gal-3 or a nucleic acid sequence sharing at least 95% identity with the nucleic acid sequence that encodes Gal-3.

In one embodiment, the subject invention provides a method for the prevention of melanoma, preferably, melanoma metastasis, comprising administering to a subject in need a composition comprising 1) a nucleic acid sequence that encodes galectin-3 (Gal-3) or a nucleic acid sequence sharing at least 95% identity with the nucleic acid sequence that encodes Gal-3, 2) a nucleic acid sequence that encodes an amino acid sequence sharing at least 95% identity with Gal-3, and/or 3) a vector comprising a nucleic acid sequence that encodes Gal-3 or a nucleic acid sequence sharing at least 95% identity with the nucleic acid sequence that encodes Gal-3, or a nucleic acid sequence that encodes an amino acid sequence sharing at least 95% identity with Gal-3.

In one embodiment, the subject invention provides a method for slowing the growth of melanoma cells, preferably, metastatic melanoma cells, the method comprising contacting the melanoma cells with a composition comprising 1) a nucleic acid sequence that encodes Gal-3 or a nucleic acid sequence sharing at least 95% identity with the nucleic acid sequence that encodes Gal-3, 2) a nucleic acid sequence that encodes an amino acid sequence sharing at least 95% identity with Gal-3, and/or 3) a vector comprising a nucleic acid sequence that encodes Gal-3 or a nucleic acid sequence sharing at least 95% identity with the nucleic acid sequence that encodes Gal-3, or a nucleic acid sequence that encodes an amino acid sequence sharing at least 95% identity with Gal-3.

In one embodiment, the subject invention provides a method for improving survival of a metastatic melanoma patient, the method comprising administering to the metastatic melanoma patient a composition comprising 1) a nucleic acid sequence that encodes Gal-3 or a nucleic acid sequence sharing at least 95% identity with the nucleic acid sequence that encodes Gal-3, 2) a nucleic acid sequence that encodes an amino acid sequence sharing at least 95% identity with Gal-3, and/or 3) a vector comprising a nucleic acid sequence that encodes Gal-3 or a nucleic acid sequence sharing at least 95% identity with the nucleic acid sequence that encodes Gal-3, or a nucleic acid sequence that encodes an amino acid sequence sharing at least 95% identity with Gal-3.

In one embodiment, the subject invention provides a method for inhibiting, suppressing, or slowing down melanoma metastasis in a subject, the method comprising administering to the subject in need a pharmaceutical composition comprising 1) a nucleic acid sequence that encodes Gal-3 or a nucleic acid sequence sharing at least 95% identity with the nucleic acid sequence that encodes Gal-3, 2) a nucleic acid sequence that encodes an amino acid sequence sharing at least 95% identity with Gal-3, and/or 3) a vector comprising a nucleic acid sequence that encodes Gal-3 or a nucleic acid sequence sharing at least 95% identity with the nucleic acid sequence that encodes Gal-3, or a nucleic acid sequence that encodes an amino acid sequence sharing at least 95% identity with Gal-3.

In a specific embodiment, the melanoma is resistant to immune checkpoint inhibitor (ICI) therapies. In a specific embodiment, the subject has been treated with an ICI therapy. In a specific embodiment, the melanoma cells are metastatic melanoma cells. In a specific embodiment, the melanoma cells are resistant to one or more ICIs.

In one embodiment, the composition is administered prior to, simultaneously with, or after the administration of the ICI therapy.

In certain embodiments, the pharmaceutical composition further comprises an inhibitor that inhibits the level of extracellular Gal-3, the binding of extracellular Gal-3 to its ligand, and/or the secretion of Gal-3 outside of the cells. In specific embodiments, the inhibitor may be, for example, an antibody to Gal-3 or an antibody to Gal-3's ligand; a competitive inhibitor that competes with Gal-3 for its ligand; and/or a non-competitive inhibitor that blocks the binding of Gal-3 to its ligand.

In one embodiment, the method of the subject invention further comprises administering to the subject a composition comprising an inhibitor that inhibits the level of extracellular Gal-3, the binding of extracellular Gal-3 to its ligand, and/or the secretion of Gal-3 outside of the cells.

### BRIEF DESCRIPTION OF DRAWINGS

The patent or application file contains at least one drawing executed in color. Copies of this patent or patent application publication with color drawing(s) will be provided by the Office upon request and payment of the necessary fec.

FIGS. 1A-1G. Tumor-intrinsic Gal-3 is downregulated from primary to metastatic melanomas. Transcriptional expression analysis of Gal-3 in 10,284 samples from 33 different types of cancer collected in the TCGA database. Each green dot represents one patient. Medians are depicted as horizontal black lines (A1). Transcriptional expression analysis of Gal-3 in 471 TCGA melanoma samples versus 812 GTEx normal skin tissues (1B). Transcriptional expression analysis of Gal-3 in metastatic melanomas versus primary melanomas retrieved from TCGA SKCM database (1C), the GEO microarray datasets GSE8401 (1D), and the GEO RNA-seq dataset GSE157740 (1E). The expression levels of Gal-3 in melanoma cell lines of variable metastatic potential by immunoblotting (1F). The expression levels of Gal-3 in the highly metastatic murine melanoma cell subline (B16/BL6) compared with its parent line (B16) by immunoblotting, RT-PCR, and flow cytometry (1G). SKCM: Skin cutaneous melanoma. (***p<0.001, **p<0.01, *p<0.05)

FIGS. 2A-2D. Gal-3 is elevated in the sera of melanoma patients. ELISA-measured serum Gal-3 levels in melanoma patients compared to age-matched healthy subjects (2A). Serum Gal-3 levels in normal individuals, patients with non-metastatic melanoma and those with metastatic melanoma (2B). scRNA-seq data analysis of immune cells within human melanoma samples retrieved from the GEO datasets GSE139249 (2C) and GSE123139 (2D). The distribution of various cells in melanoma TME within each dataset is depicted in the upper panels. The middle panels represent heatmaps of Gal-3 expression levels in the TME cells within the corresponding datasets. The lower panels represent quantification of Gal-3 expression data, categorized based on cell type. (***p<0.001)

FIGS. 3A-3J. Loss of Gal-3 enhances the metastatic behavior of melanoma cells. Gal-3 silencing in SK-MEL-2 (3A) and G361 (3B) melanoma cells using three different shRNA constructs as examined by immunoblotting. Effect of Gal-3 knockdown within SK-MEL-2 and G361 melanoma cells on cell morphology (3C), migration and invasion (3D and 3E), colony formation (3F and 3G), and in vivo xenograft formation abilities (3H and 3I). Intravenously injected luciferase labelled G361 ScrCtrl and Gal3KD cells into NSG mice as monitored by the in vivo imaging system at 0, 1, 2, 3, 4, and 5 weeks after the injection (3J, upper panel). Average tumor growth rates as measured by photon flux in G361 ScrCtrl.Luc and Gal3KD.Luc injected mice (3J, lower panel). The photon counts are indicated by the color scale. (***p<0.001, **p<0.01, *p<0.05)

FIGS. 4A-4I. Intracellular Gal-3 regulates the activities of signaling pathways and NFAT1 in melanoma cells. Effect of Gal-3 silencing within SKMEL2 (4A) and G361 (4B) melanoma cells and Gal-3 overexpressing within A375 melanoma cells (4C) on the activity of the Ras/MAPK and PI3K/Akt signaling pathways as examined by immunoblotting. Transcriptional expression analysis of NFAT1 in human metastatic melanomas versus primary melanomas retrieved from TCGA SKCM database (4D). The expression levels of NFAT1 in the highly metastatic murine melanoma cell subline (B16/BL6) compared with its parent line (B16) by RT-PCR (4E). Spearman's correlation analysis between the expression levels of Gal-3 and NFAT1 in TCGA SKCM database, represented as a scatter plot (4F). NFAT1 expression analysis in the three established Gal-3-silenced SK-MEL-2 lines (Gal3KD #1, Gal3KD #2, and Gal3KD #3) compared with the scrambled control cells as examined by immunoblotting (4G). NFAT1 expression analysis in Gal-3-silenced G361 melanoma cells compared with the scrambled control cells by immunoblotting (4H). NFAT1 expression analysis in Gal-3-overexpressing A375 melanoma cells compared with empty vector control cells by immunoblotting (4I). (***p<0.001, **p<0.01, *p<0.05)

FIG. 5. Proposed role of tumor-intrinsic Gal-3 in regulating melanoma metastasis. Gal-3 is expressed at high levels in normal human melanocytes and melanoma cells in primary cutaneous lesions (Left) and progressively lost as melanoma cells enter the circulation and form metastases (Right). We propose that diminished intracellular Gal-3 levels result in the unleashing of NFAT1 expression that facilitates dislodging of melanoma cells from the primary tumor and migration through the bloodstream to initiate metastatic colonization in distal organs.

FIGS. 6A-6C. Gal-3 silencing in SK-MEL-2 (6A) and G361 (6B) melanoma cells using three different shRNA constructs as examined by RT-PCR. (***p<0.001). Gal-3 silencing in SK-MEL-2 melanoma cells was performed using three different shRNA constructs, then validated by flow cytometry (6C).

FIGS. 7A-7D. In vitro assay of luciferase activity in serially diluted SK-MEL-2 (7A) and G361 (7B) Scr.Luc and Gal-3KD.Luc cells versus their corresponding controls following puromycin selection. The photon counts are indicated by the color scale. Average tumor growth rates as measured by photon flux in SK-MEL-2 ScrCtrl.Luc and Gal3KD.Luc injected mice (7C). Representative pictures for livers, lungs, and brains of sacrificed G361 ScrCtrl.Luc and Gal3KD.Luc injected mice (7D). Arrows indicate melanoma metastases. (***p<0.001)

FIGS. 8A-8G. Enforced Gal-3 expression in A375 melanoma cells as examined by immunoblotting (8A) and RT-PCR (8B). NFAT1 expression analysis in Gal-3-silenced SK-MEL-2 (8C), Gal-3-silenced G361 (8D), and Gal-3-overexpressing A375 (8E) melanoma cells as examined by RT-PCR. Effect of Gal-3 silencing within SKMEL2 melanoma cells on the expression levels of MMP-3 (8F), and IL-8 (8G) by RT-PCR. (***p<0.001, **p<0.01, *p<0.05)

FIGS. 9A-9C. Gal-3 expression in normal human epidermal melanocytes (nHEM) versus a panel of human melanoma cell lines (A375, A2058, SK-MEL-2 and SK-Mel-5) using RT-qPCR (9A) and immunoblotting (9B). The expression levels of Gal-3 in SK-MEL-2, SK-MEL-5, A375, and A2058 melanoma cell lines versus nHEM as examined by flow cytometry (9C). (**p<0.01)

FIGS. 10A-10C. Effect of Gal-3 silencing within SKMEL2 melanoma cells on the activity of the Wnt/β-catenin (10A-10C) signaling pathways as examined by immunoblotting.

FIGS. 11A-11C. GPC6 is a potential target of Gal-3-NFAT1 regulatory axis in melanoma. Effect of Gal-3 silencing within SKMEL2 melanoma cells on the expression levels of GPC6 by RT-PCR (11A). Transcriptional expression analysis of GPC6 in human metastatic melanomas versus primary melanomas retrieved from TCGA SKCM database (11B). Spearman's correlation analysis between GPC6 and Gal-3 (r=−0.2838, P<0.001) (c, heatmap and right upper panel) or between GPC6 and NFAT1 (r=0.2557, P<0.001) (11C, heatmap and right lower panel) in the TCGA melanoma data. (***p<0.001, **p<0.01)

### BRIEF DESCRIPTION OF SEQUENCES

SEQ ID NOs: 1-3 are shRNA target sequences of human LGALS3 shRNA contemplated for use according to the subject invention.

SEQ ID NO: 4 is a non-target sequences of human LGALS3 shRNA scrambled control contemplated for use according to the subject invention.

SEQ ID NO: 5 is an amino acid sequence of Gal-3 protein contemplated for use according to the subject invention.

SEQ ID NO: 6 is a coding DNA sequence of human LGALS3 contemplated for use according to the subject invention.

### DETAILED DESCRIPTION

The present invention provides methods and composition for diagnosis, prognosis, prevention and/or treatment of cancers such as melanomas, in particular metastatic melanoma. The subject invention provides biomarkers and methods for assessing the severity of a cancer/tumor and for monitoring the progressing of a cancer/tumor. The subject invention also provides compositions for treating a cancer/tumor, and for preventing or reducing the progression of a cancer/tumor. The compositions according to the subject invention regulate malignancy-associated pathways and alter melanoma signaling, growth, and survival.

In one embodiment, the subject invention identifies biomarkers that are involved in cancer metastasis. In a specific embodiment, the subject invention identifies Gal-3 as a metastasis-suppressive molecule in melanoma. In one embodiment, the subject invention identifies the tumor-suppressive function of Gal-3 in melanoma cells and, importantly, identifies intracellular Gal-3 as a prognostic indicator of metastatic risk in melanoma patients.

Advantageously, the subject invention helps meet the demand caused by the lack of biomarkers of melanoma metastasis and offers an immunohistopathological marker to assay, whose presence (i.e., expression) in melanoma cells is indicative of a non-metastatic state and its absence is indicative of high metastatic potential and triggers more aggressive treatment approaches.

In one embodiment, the cancers exhibit significant transcriptional changes in glycosylation-related genes. In a specific embodiment, the cancer is a skin cancer such as melanoma, preferably, metastatic melanoma (MM). Melanoma is one of the most aggressive forms of cancer, typically beginning in the skin and often metastasizing to vital organs and other tissues. Melanomas include, but are not limited to, superficial spreading melanoma (SSM), nodular melanoma (NM), Lentigo maligna, lentigo maligna melanoma (LMM), mucosal melanoma, polypoid melanoma, desmoplastic melanoma, amelanotic melanoma, soft-tissue melanoma, uveal melanoma and acral lentiginous melanoma (ALM).

In certain embodiments, the melanoma may be a stage 0, I, II, III or IV melanoma. Stage 0 melanoma is a very early stage disease known as melanoma in situ. The tumor is limited to the epidermis with no invasion of surrounding tissues, lymph nodes, or distant sites. Stage 0 melanoma is considered to be very low risk for disease recurrence or spread to lymph nodes or distant sites.

Stage I melanoma is characterized by tumor thickness, presence and number of mitoses, and ulceration status. Stage I melanomas are considered to be low-risk for recurrence and metastasis. Sentinel lymph node biopsy is recommended for Stage I tumors thicker than 1.0 mm and for any ulcerated tumors of any thickness. Surgery is a common treatment for Stage I melanoma.

Stage II melanomas also are localized tumors characterized by tumor thickness and ulceration status. Stage II melanoma is considered to be intermediate-risk for local recurrence or distant metastasis. In addition to biopsy and surgery as described for Stage I, Stage II treatment may include adjuvant therapy, which is a treatment given in addition to a primary cancer treatment, following surgery. Treatments may include interferons therapies (e.g., interferon alfa-2a, and/or alfa-2b), and vaccine therapy.

Stage III melanomas are tumors that have spread to regional lymph nodes, or have developed in transit metastasis or satellites. Stage III disease is considered to be intermediate-to high-risk for local recurrence or distant metastasis. In addition to surgery and adjuvant therapy as described above, Stage III melanoma treatment often includes therapeutic lymph node dissection (TLND) to remove regional lymph nodes from the area where cancerous lymph nodes were found. The goal of the surgery is to prevent further spread of the disease through the lymphatic system.

Stage IV melanomas often are associated with metastasis beyond the regional lymph nodes to distant sites in the body. Common sites of metastasis are vital organs (lungs, abdominal organs, brain, and bone) and soft tissues (skin, subcutaneous tissues, and distant lymph nodes). Stage IV melanoma may be characterized by the location of the distant metastases; the number and size of tumors; and the serum lactate dehydrogenase (LDH) level. Elevated LDH levels usually indicate that the tumor has spread to internal organs. Treatments may include surgery to remove cancerous tumors or lymph nodes that have metastasized to other areas of the body, systemic therapies and radiation therapy.

Visual diagnosis of melanomas is still the most common method employed by health professionals. Metastatic melanomas can be detected by X-rays, CT scans, MRIs, PET and PET/CTs, ultrasound, LDH testing and photoacoustic detection.

The subject invention further provides methods and compositions for inhibiting the growth of primary melanomas, inhibiting metastasis, inhibiting the growth of metastases, killing circulating melanoma cells, inducing remission, extending remission, and/or inhibiting recurrence.

In one embodiment, the subject invention pertains to the identification of Gal-3 as being involved in the pathogenesis of melanomas, e.g., MM. In certain embodiments, the methods according to the subject invention use Gal-3 as a biomarker for cancer diagnosis, progression and/or metastasis, for example: (1) the diagnosis of cancer; (2) the prognosis of cancer (e.g., monitoring cancer progression or regression from one biological state to another); (3) the susceptibility or prediction of response to treatment for a cancer; (4) the metastasis of cancer; and/or (5) the evaluation of the efficacy to a treatment for a cancer.

For the diagnosis of a cancer, the level of the specific biomarker in a subject or a sample of the subject can be compared to a baseline or control level. If the level is below or above the control level, a certain cancer is implicated. The prognosis of a cancer can be assessed by comparing the level of the specific biomarker at a first time point to the level of the biomarker at a second time point that occurs at a given interval. The prediction of response to treatment for a cancer can be determined by obtaining the level of a specific biomarker and correlating this level to a standard curve. The evaluation of the efficacy of the treatment for a cancer can be assessed by comparing the level of the specific biomarker before administration of the treatment to the level of the biomarker after the administration of the treatment.

Expression of genes of the present invention can be measured by many methods known in the art. In general, expression of a nucleic acid molecule (e.g., RNA or DNA) can be detected by any suitable method or technique of measuring or detecting gene or polynucleotide sequence or expression. Such methods include, but are not limited to, polymerase chain reaction (PCR), reverse transcriptase PCR (RT-PCR), in situ PCR, quantitative PCR (q-PCR), in situ hybridization, flow cytometry, Western blot, Southern blot, Northern blot, immunohistochemistry, sequence analysis, microarray analysis, mass spectrometry analysis, detection of a reporter gene, or any other DNA/RNA hybridization platforms.

### Gal-3

Gal-3 (previously known as Mac-2 antigen) is the only member in the chimeric galectin group. It is a 29-35 kDa protein. The unique chimeric structure of Gal-3 accounts for its distinguished biological roles among other galectin members. Gal-3 has an unfolded N-terminal domain (NTD; residues 1-111) linked to a globular C-terminal carbohydrate-recognition domain (CRD; residues 112-250).

The N-terminal domain consists of a 21-amino-acid N-terminal segment (NTS), harboring two serine phosphorylation sites, and nine collagen-like repeats rich in proline and glycine. The NTS sequence is essential for Gal-3 nucleocytoplasmic translocation and secretion. The C-terminal domain of Gal-3 has a 5-stranded β-sheet (F-face; F1-F5) in addition to the canonical 6-stranded β-sheet (S-face; S1-S6), both together form the CRD β-sandwich via intramolecular antiparallel folding. The concave side of the β sandwich with its defined five subsites (A-E) accommodates the bound glycan. Of note, the conserved subsite C docks the β-galactose, while the adjacent residue occupies subsite D, forming the disaccharide-binding site. Other less conserved subsites are accountable for the variable specificity of individual galectins to longer glycans.

Unlike other members of the galectin family, Gal-3 has a distinctive Asp-Trp-Gly-Arg (NWGR) motif within its C-terminal domain (residues 180-183). Interestingly, this motif is homologous to the anti-death motif of the Bel-2 family, which could explain, in part, the anti-apoptotic activity of cytoplasmic Gal-3.

Generally, Gal-3 exists as monomers in solution. In the absence of glycosylated ligands, Gal-3 can homodimerize via its CRDs, while Gal-3 binding to its glycoprotein or glycolipid ligand triggers spontaneous oligomerization up to pentamers through NTD interactions in a dynamic process similar to liquid-liquid phase separation (LLPS). Gal-3 exhibits the pentameric structure exclusively outside of cells, where its formation is largely dependent on Gal-3 concentration as well as interacting glycoconjugate type and concentration. Such extracellular arrangement forms a lattice-like structure by cross-linking between adjacent cells or between cells and extracellular matrix. Gal-3 binding to cell surface glycosylated proteins such as integrins and epidermal growth factor receptors (EGFR), and extracellular matrix proteins like laminin and fibronectin plays vital roles in regulation of various extracellular biological processes.

Mechanistically, both NTD and CRD are involved in Gal-3 polymerization. Gal-3 aggregation is initiated by accumulating interactions between NTD's aromatic residues, namely tryptophan and tyrosine. These interactions are augmented by Gal-3-glycan binding and the evolving high local protein concentration, while they are blocked by lactose that interferes with Gal-3 binding to its ligand. Intramolecular interactions between N-terminal proline residues and the F-face of the CRD are directly implicated in glycan-binding and subsequent Gal-3 polymerization.

The human Gal-3 gene (LGALS3) is located on chromosome 14, locus q21-922. The gene spans approximately 17 kilobases and consists of six exons separated by five introns. Multiple regulatory sequences are located within its promoter region including: two nuclear factor-κB (NF-κB)-like sites (between -229 and -105) and five cAMP-dependent response element (CRE) motifs (between -836 and -513), suggesting that the transcription factors NF-κB and cAMP-response element binding factor (CREB) function as a potential regulators of Gal-3 gene expression. Additionally, five GC box motifs for binding of Sp1 transcription factor are present within the LGALS3 promoter, which is often associated with the promoters of housekeeping genes.

Gal-3 expression is conspicuously dependent upon local oxygen tension. Using HeLa cells and mouse embryonic fibroblasts (MEFs) exposed to hypoxic conditions, the data show that hypoxia inducible factor (HIF)-1α interacts with hypoxia regulatory elements in the Gal-3 promoter region to upregulate Gal-3 expression.

Gal-3 is expressed intracellularly, in cytoplasm, nucleus and mitochondria, where it participates in the regulation of cell proliferation, cell cycle, and apoptosis through modulation of many signaling pathways. It is also secreted out of the cell where it largely modulates cell adhesion and immune surveillance. Certain amino acid residues in the Gal-3 polypeptide chain control the directional movement of Gal-3 through the nuclear pore complex (NPC) via a receptor-mediated system. A nuclear localization signal (NLS), analogous to that of c-Myc and p53 NLSs, is considered essential for Gal-3 translocation into the nucleus mediated via the importin-α/β heterodimer, while a leucine-rich nuclear export signal (NES) motif is thought to be crucial for Gal-3 translocation into the cytoplasm carried by the chromosomal region maintenance/exportin 1/Xpol (CRM1). Both NLS and NES were recognized near the C-terminus of the Gal-3 polypeptide chain with an overlapping sequence.

Gal-3, like other members of the galectin family, is characterized by a lack of an amino-terminal signal peptide sequence, which is important to route cellular proteins via the classical secretion pathway. This implies that Gal-3 released into the extracellular space follows a non-classical secretion pathway, independent of the endoplasmic reticulum (ER)-Golgi complex.

Asparagine (N)-linked-glycosylation is a post-translational protein modification that undergoes structural revision by cells undergoing adaption to various external stimuli, and accordingly facilitates regulation of cellular functions, such as cell proliferation, migration and differentiation. Altered surface N-linked glycans, which are characteristic in cancer, often correlate with cancer progression and metastasis. Increased branching of N-linked glycans expressed on surface glycoproteins enhances Gal-3—N-linked glycan interactions, which in turn, regulate diverse activities of cancer and immune cells. Gal-3 binds preferentially to internal linear N-acetyllactosamine (LacNAc) units within a linear poly-LacNAc chain that can be found on the branched N-linked glycans, while the presence of other modifications in these poly-LacNAcs can reduce or inhibit this binding.

In certain embodiments, the Gal-3 binding glycoproteins include, but are not limited to, integrins, lysosome-associated membrane proteins (LAMPs), melanoma cell adhesion molecule (MCAM/MUC-18/CD146), basigin (CD147), chondroitin sulfate proteoglycan 4 (CSPG4), intercellular adhesion molecule 1 (ICAM-1/CD54), and Mac-2BP (Mac-2-binding protein/LGALS3BP).

In specific embodiments, the Gal-3 binding glycoproteins include, for example, integrin subunit α3; integrin subunit α5; integrin subunit α6; integrin subunit β1; LAMP-1 (CD107a); LAMP-2 (CD107b); MCAM (CD146); Basigin (CD147); intercellular adhesion molecule 1 (ICAM-1/CD54); Tetraspanin-30, LAMP-3 (CD63); and CD44.

Importantly, the human enzyme I-branching β1,6-N-acetylglucosaminyl transferase 2, encoded by the GCNT2 gene, which forms I-branched (-3Galβ1,4GlcNAcβ1,3 (Galβ1,4GlcNAcβ1,6) Galβ1-) poly-LacNAc chains, decrease Gal-3-binding. Increased abundance of linear poly-LacNAcs due to reduced expression of the GCNT2 corresponds with human melanoma progression and human melanoma xenograft growth, colony formation and cell survival. These melanoma glycome traits increase Gal-3 binding to melanoma surface glycosylated proteins.

Through its interactions with intracellular protein partners and extracellular glycosylated ligands, Gal-3 can either facilitate or impair diverse biological processes associated with tumor growth and metastasis, such as intracellular proliferative and apoptotic signaling, cell-cell and cell-matrix interactions, tumor immune surveillance, and metastasis to distal tissues.

In certain embodiments, the subject invention utilizes the pro-apoptotic activity of intracellular Gal-3. Specifically, nuclear localization is usually associated with pro-apoptotic activity where Gal-3 exerts its action through its interaction with components of the intrinsic and extrinsic apoptotic pathways.

Binding of Gal-3 lattices to glycosylated cell surface receptors have a dominant role in controlling cell aggregation and cell-matrix adhesion through modulating receptor trafficking and clustering. Binding of Gal-3 lattices to cell surface receptors, such as EGF and TGF-β receptors, limits the internalization and degradation of these receptors, and thus boosts downstream cytokine signaling cascade. Gal-3 plays a dual role in enhancing distant melanoma metastasis, not only in the primary tumor site, but also in the metastatic site, and this is probably dictated by the glycan profile of the tumor cells.

In the present invention, Gal-3 expression (e.g., extracellular and/or intracellular expression) was explored in normal skin tissue, melanoma tissues (e.g., primary, circulating, and metastatic melanomas), melanoma patient sera, as well as murine melanoma models to examine its causal role in metastatic behavior. While Gal-3 was generally elevated in melanoma patient sera compared with levels in normal healthy volunteers, Gal-3 expression is downregulated in melanoma tissues compared to normal skin and reaches its lowest levels in metastatic stages (normal human melanocytes>primary melanomas>circulating melanoma cells>metastatic melanomas).

Enforced silencing of Gal-3 in melanoma cells promoted migration, invasion, colony formation, in vivo xenograft growth and metastasis, and activated canonical oncogenic signaling pathways. Also, data using Gal-3-silenced melanoma cells revealed a sustained activation of the PI3K/AKT, MAPK/ERK, and Wnt/β-catenin signaling pathways. Moreover, loss of Gal-3 in melanoma cells resulted in upregulated expression of the prometastatic transcription factor, nuclear factor of activated T cells (NFAT1). These results implicate melanoma intracellular Gal-3 as a major determinant of its metastatic behavior and reveal a negative regulatory role for Gal-3 on the expression of NFAT1 in melanoma cells.

Further data also provide evidence of the inhibitory role for intracellular Gal-3 on the expression of several metastasis-associated proteins, matrix metalloproteinase-3 (MMP-3), interleukin-8 (IL-8), and glypican-6 (GPC6), in melanoma cells. MMP-3 is a member of the matrix metalloproteinase family, responsible for the cleavage of various ECM substances, such as type IV collagen, laminin, and E-cadherin, making it a significant player in driving tumor invasion. IL-8 is a pro-inflammatory cytokine that mediates tumor metastasis via facilitating invasion, migration, and angiogenesis. GPC6 is a cell-surface proteoglycan that enhances proliferation and invasion of tumor cells in many cancer types. Thus, the metastasis-suppressive function of Gal-3 in melanoma cells is probably mediated via not only Gal-3-dependant downregulation of NFAT1 but also its metastasis-associated downstream targets MMP-3, IL-8, and GPC6.

In one embodiment, the subject invention provides an inhibition of Gel-3 expression using gene silencing technologies, e.g., siRNAs or shRNAs targeting Gal-3. In specific embodiments, the shRNA targeting Gal-3 is placed in a construct, e.g., a viral plasmid, wherein the construct comprises a DNA sequence that is transcribed into an shRNA targeting Gal-3. In a specific embodiment, the DNA sequence for the shRNA targeting Gal-3 comprises the sequence of SEQ ID NO: 1, 2 or 3.

In certain embodiments, the shRNA comprises a sequence fully complementary or substantially complementary (e.g., at least 80%, 85%, 90%, 95% or 100% complementary) to a sequence in a target gene. In some embodiments, the shRNA targeting Gal-3 comprises a sequence fully complementary or substantially complementary (e.g., at least 80%, 85%, 90%, 95% or 100% complementary) to a target sequence of Gal-3. In a specific embodiment, the shRNA targeting Gal-3 comprises a sequence fully complementary or substantially complementary (e.g., at least 80%, 85%, 90%, 95% or 100% complementary) to SEQ ID NO: 1, 2 or 3.

As used herein, the term “fully complementary” with regard to a sequence refers to a complement of the sequence by Watson-Crick base pairing, whereby guanine (G) pairs with cytosine (C), and adenine (A) pairs with either uracil (U) or thymine (T). A sequence may be fully complementary to the entire length of another sequence, or it may be fully complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence.

As used herein, the term “substantially complementary” refers to sequences of nucleotides where a majority (e.g., at least about 80%, 85%, 90%, 95%, 96%, 97%, 98%, or 99%) or all of the bases in the sequence are complementary, or one or more (e.g., no more than 20%, 15%, 10%, 5%, 4%, 3%, 2%, or 1%) bases are non-complementary, or mismatched. A complementary sequence can be a reverse complement of the sequence allowing for Watson-Crick base pairing, wobble base pairing, or both, whereby G pairs with either C or U, and A pairs with either U or T. A sequence may be complementary to the entire length of another sequence or it may be complementary to a specified portion or length of another sequence. One skilled in the art will recognize that the U may be present in RNA, and that T may be present in DNA. Therefore, a U within an RNA sequence may pair with A or G in either an RNA sequence or a DNA sequence, while an A within either of an RNA or DNA sequence may pair with a U in a RNA sequence or T in a DNA sequence. Two sequences that are substantially complementary may hybridize to each other, e.g., under low stringency, medium stringency, high stringency, or very high stringency conditions.

As used herein, the term “construct,” “expressing construct” or “expression construct” is a generic term that includes nucleic acid preparations designed to achieve an effect of interest. An expressing construct comprises an RNAi molecule that can be cleaved in vivo to form an siRNA or a mature shRNA. For example, an RNAi construct is an expression vector capable of giving rise to an siRNA or a mature shRNA in vivo.

In one embodiment, the subject invention identifies Gal-3 as a metastasis-suppressive molecule in melanoma.

In one embodiment, the nucleic acid sequence of Gal-3 gene (LGALS3) comprises, or consists of, a sequence of SEQ ID NO: 6, or a nucleic acid sequence sharing at least 50%, 55%, 60%, 65%, 70%, 75%, 80%, 85%, 90%, 95%, 96%, 97%, 98% or 99% identity with SEQ ID NO: 6.

In one embodiment, the amino acid sequence of Gal-3 protein comprises, or consists of, a sequence of SEQ ID NO: 5 or an amino acid sequence sharing at least 50%, 55%, 60%, 65%, 70%, 75%, 80%, 85%, 90%, 95%, 96%, 97%, 98% or 99% identity with SEQ ID NO: 5.

In one embodiment, the nucleic acid sequence of Gal-3 comprises, or consists of, a nucleic acid sequence that encodes SEQ ID NO: 5 or an amino acid sequence sharing at least 50%, 55%, 60%, 65%, 70%, 75%, 80%, 85%, 90%, 95%, 96%, 97%, 98% or 99% identity with SEQ ID NO: 5.

### Compositions

In one embodiment, the subject invention provides a pharmaceutical composition comprises:
1) a nucleic acid sequence that encodes Gal-3 or a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3;
2) an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3;
3) a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
4) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
5) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
6) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the composition of the subject invention further comprises a therapeutic agent for treating the cancer, such as melanoma.

In one embodiment, the subject invention provides a pharmaceutical composition comprising an inhibitor for inhibiting extracellular Gal-3's polymerization, the level of extracellular Gal-3, the binding of extracellular Gal-3 to its ligands, and the secretion of Gal-3 outside of cells. In specific embodiments, the inhibitor may be, for example, an antibody to Gal-3 or an antibody to Gal-3's ligand; a competitive inhibitor that competes with Gal-3 for its ligand; and/or a non-competitive inhibitor that blocks the binding of Gal-3 to its ligand. In a specific embodiment, the inhibitor is GB1211.

In certain embodiments, the composition of the subject invention comprises:
A) at least one of:
   1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
   2) an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3;
   3) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
   4) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
   5) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3; and
B) an inhibitor for inhibiting extracellular Gal-3's polymerization, the level of extracellular Gal-3, the binding of extracellular Gal-3 to its ligands, and the secretion of Gal-3 outside of cells.

In one embodiment, the composition according to the subject invention also comprises a pharmaceutically acceptable carrier. “Pharmaceutically acceptable carrier” refers to a diluent, adjuvant or excipient with which the active ingredient disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith. Examples of carriers suitable for use in the pharmaceutical compositions are known in the art and such embodiments are within the purview of the invention.

The compositions of the present invention can be administered to the subject being treated by standard routes, including the local, oral, ophthalmic, nasal, topical, intratumoural, transdermal, intra-articular, parenteral (e.g., intravenous, intraperitoneal, intradermal, subcutaneous or intramuscular), intracranial, intracerebral, intraspinal, intravaginal, intrauterine, or rectal route. Additionally, the composition may be administered directly into the tumor of melanoma. Depending on the condition being treated, one route may be preferred over others, which can be determined by those skilled in the art.

In one embodiment, the composition is formulated in accordance with routine procedures as a pharmaceutical composition adapted for local administration to human beings. Typically, compositions for local administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile water for injection or saline can be provided so that the ingredients may be mixed prior to administration.

In one embodiment, the pharmaceutical composition of the subject invention may further comprise one or more therapeutic agents. The therapeutic agent may comprise a chemotherapeutic agent (e.g., dacarbazine or cisplatin), immunotherapeutic agent (e.g., interleukin-2 (IL-2) or interferon (IFN)), gene therapy and/or radio therapeutic agent. The therapeutic agent may further comprise other cytotoxic agents such as anti-tumour peptides, cytokines e.g., IFN-γ, TNF, CSF and growth factors, and/or cancer vaccines.

In one embodiment, dosage units containing the nucleic acid and/or peptidic molecules contain about 0.01 mg to 1000 mg, about 0.01 mg to 900 mg, about 0.01 mg to 800 mg, about 0.01 mg to 700 mg, about 0.01 mg to 600 mg, about 0.01 mg to 500 mg, about 0.05 mg to 500 mg, about 0.1 mg to 400 mg, about 0.1 mg to 300 mg, about 0.1 mg to 200 mg, about 0.1 mg to 100 mg, about 0.1 mg to 90 mg, about 0.1 mg to 80 mg, about 0.1 mg to 70 mg, about 0.1 mg to 60 mg, about 0.1 mg to 50 mg, about 0.1 mg to 40 mg, about 0.1 mg to 30 mg, about 0.1 mg to 20 mg, about 0.1 mg to 10 mg, about 0.5 mg to 50 mg, about 1 mg to 40 mg, about 1 mg to 20 mg, about 1 mg to 10 mg, or about 1 mg to 5 mg.

In one embodiment, the composition may be formulated for administration as tablets, coated tablets, nasal sprays, solutions, emulsions, liposomes, powders, capsules or sustained release forms.

In specific embodiments, the composition of the subject invention may be administered at least once a day, twice a day, or three times a day for consecutive days, e.g. 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. The composition of the subject invention may also be administered for weeks, months or years.

Depending on the form of the pharmaceutical composition and/or mode of administration of the present invention, pharmaceutically acceptable carriers may include, but are not limited to, pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid, or talc. If desired and suitable, a coating material may also be used such as glyceryl monostearate or glyceryl distearate, for example, to delay absorption in the gastrointestinal tract if the pharmaceutical composition is in the form of a solid form.

In one embodiment, the pharmaceutical composition may be formulated for parenteral administration (e.g., by injection, for example bolus injection or continuous infusion). In addition, the composition may be presented in unit dose form in ampoules, pre-filled syringes, and small volume infusion or in multi-dose containers with or without an added preservative. The composition may be in forms of suspensions, solutions, or emulsions in oily or aqueous vehicles. The composition may further contain formulation agents such as suspending, stabilizing and/or dispersing agents. In a further embodiment, the active ingredient of the composition according to the invention may be in a powder form, obtained by aseptic isolation of sterile solid or by lyophilization from solution for constitution with a suitable vehicle, e.g., sterile, pyrogen-free water, before use.

In one embodiment, the composition may be formulated in aqueous solutions for oral administration. The composition may be dissolved in suitable solutions with added suitable colorants, flavors, stabilizing and thickening agents, artificial and natural sweeteners, and the like. In addition, the composition may further be dissolved in solution containing viscous material, such as natural or synthetic gums, resins, methylcellulose, sodium carboxymethylcellulose, or other well-known suspending agents.

In one embodiment, the composition is applied topically or systemically or via a combination of both. The composition may be formulated in the forms of lotion, cream, gel, and the like.

In one embodiment, the composition can be applied directly to the nasal cavity by conventional means, for example with a dropper, pipette, or spray. The compositions may be provided in single or multi-dose form. Administration to the respiratory tract may also be achieved by means of an aerosol formulation in which the active ingredient is provided in a pressurized pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin.

Furthermore, the composition may be provided in the form of a dry powder in a suitable powder base such as lactose, starch, starch derivatives such as hydroxypropylmethyl cellulose and polyvinylpyrrolidone (PVP). Conveniently, the powder carrier will form a gel in the nasal cavity. The powder composition may be presented in unit dose form for example in capsules or cartridges of, e.g., gelatin, or blister packs from which the powder may be administered by means of an inhaler.

In a further embodiment, the pharmaceutical composition according to the present invention is a sustained release system such as semipermeable matrices of solid hydrophobic polymers containing the active ingredient of the present invention. In another embodiment, the pharmaceutical composition is in a liquid form such as aqueous or non-aqueous solutions, suspensions, emulsions, elixirs, and capsules filled with the same.

### Therapeutic Applications

In one embodiment, the subject invention provides methods for monitoring the metastatic behavior of melanoma. The methods of the subject invention use Gal-3 as the biomarker for diagnosing a cancer and/or assessing cancer metastasis and progression, and for negatively regulating pro-metastasis factors, e.g., NFAT1 as well as metastasis-associated proteins.

In one embodiment, the subject invention provides methods for treating a cancer, e.g., melanoma, preferably, MM, which involve the reduction of extracellular level of Gal-3, the inhibition of extracellular Gal-3 function, and/or the increase of the intracellular Gal-3 expression.

The present invention reveals that, as melanoma cells lose galectin-3 expression, they gain virulent features that prime metastasis. These findings implicate the prognostication value of melanoma cell galectin-3 as an indicator of melanoma progression. These results highlight a tumor-suppressive function of Gal-3 in melanoma cells, implicating intracellular Gal-3 as a prognostic indicator of metastatic risk in melanoma patients.

Based on the various mechanisms through which Gal-3 contributes to tumor development and progression, blocking Gal-3 with specific inhibitors can be used to impede tumor progression and metastasis. For example, modified citrus pectin (MCP) is a complex polysaccharide of D-galacturonic acid residues linked together by alpha-1, 4 glycosidic bonds. By antagonizing Gal-3 action, MCP slows tumor growth, inhibits its metastasis and strengthens anti-tumor immunity. Additionally, the combination of MCP and the photosensitizer chlorin e6 (Ce6) can be used for melanoma management.

In one embodiment, the present invention provides methods that target the intracellular Gal-3 protein expression and function to slows tumor growth, inhibits its metastasis and strengthens anti-tumor immunity.

In one embodiment, the subject invention provides methods for treating a cancer, such as melanoma, e.g., MM, in a subject. In a specific embodiment, the method comprises:
(i) assessing the expression level of intracellular Gal-3 in a sample obtained from the subject;
(ii) comparing the expression level of intracellular Gal-3 in the sample to a reference derived from the expression level of Gal-3 in samples obtained from healthy subjects, or a baseline for said subject;
(iii) identifying the cancer, such as melanoma, e.g., MM, in the subject based on the decreased level of intracellular Gal-3 in the test sample; and
(iv) administering a treatment to the subject.

In one embodiment, the treatment to the subject may comprise administering to the subject a pharmaceutical composition according to the subject invention or a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the present invention provides methods that target the extracellular Gal-3 protein to slows tumor growth, inhibits its metastasis and strengthens anti-tumor immunity. In certain embodiments, targeting extracellular Gal-3 may involve the interference or inhibition of extracellular Gal-3's polymerization, the level of extracellular Gal-3, and the binding of extracellular Gal-3 to its ligands (e.g., integrins, epidermal growth factor receptors (EGFR), laminin and fibronectin), for example, on cells and/or extracellular matrix, and/or may involve the inhibition or reduction of the secretion of Gal-3 outside of cells.

In specific embodiments, the inhibitor may be, for example, an antibody to Gal-3 or an antibody to Gal-3's ligand; a competitive inhibitor that competes with Gal-3 for its ligand; and/or a non-competitive inhibitor that blocks the binding of Gal-3 to its ligand. In a specific embodiment, the inhibitor is GB1211.

In certain embodiments, the method for treating a cancer, such as melanoma, e.g., MM, in a subject may comprise:
(i) assessing the expression level of extracellular Gal-3 in a sample obtained from the subject;
(ii) comparing the expression level of extracellular Gal-3 in the sample to a reference derived from the expression level of Gal-3 in samples obtained from a healthy subject, or a baseline for said subject;
(iii) identifying the cancer, such as melanoma, e.g., MM, in the subject based on the increased level of extracellular Gal-3 in the test sample; and
(iv) administering a treatment to the subject.

In one embodiment, the treatment may comprise administering to the subject an inhibitor that inhibits or blocks extracellular Gal-3's polymerization, the level of extracellular Gal-3, and the binding of extracellular Gal-3 to its ligands (e.g., integrins, epidermal growth factor receptors (EGFR), laminin and fibronectin), and/or inhibits or reduce the secretion of Gal-3 outside of cells.

In a further embodiment, the treatment is systemic and comprises administering Immune Checkpoint Inhibitors (ICIs), e.g., anti-PD1, anti-PDL1 and/or anti-CTLA4 treatments.

Immune checkpoints are known in the art and the term is well understood in the context of cancer therapy. Immune checkpoints include, but are not limited to, cytotoxic T-lymphocyte antigen 4 (CTLA-4), programmed cell death protein 1 (PD-1) and its ligand PDL-1, T-cell immunoglobulin and mucin domain-containing protein 3 (TIM-3), killer cell immunoglobulin-like receptor (KIR), lymphocyte activation gene-3 (LAG-3), V-domain immunoglobulin suppressor of T cell activation (VISTA), and B and T lymphocyte attenuator (BTLA). Inhibitors of immune checkpoints inhibit their normal immunosuppressive function, for example, by down regulating the expression of checkpoint molecules or by binding thereto and blocking normal receptor/ligand interactions. As a result, inhibitors of immune checkpoints enhance the immune response to an antigen, in particular, from a tumor cell.

Inhibitors of immune checkpoints are known in the art and preferred inhibitors include anti-immune checkpoint antibodies, such as anti-CTLA-4 antibodies (e.g., ipilimumab and tremelimumab), anti-PD-1 antibodies (e.g., nivolumab, lambrolozumab, pidilizumab and RG7446 (Roche)) and anti-PDL-1 antibodies (e.g., BMS-936559 (Bristol-Myers Squibb), MPDL3280A (Genentech), MSB0010718C (EMD-Serono) and MED14736 (AstraZeneca)).

With knowledge of an immune checkpoint target, a skilled artisan can administer an inhibitor thereof. Inhibitors may be selected from proteins, peptides, peptidomimetics, peptoids, antibodies, antibody fragments, small inorganic molecules, small non-nucleic acid organic molecules or nucleic acids such as anti-sense nucleic acids, small interfering RNA (siRNA) molecules or oligonucleotides. The inhibitor may for example be a modified version of the natural ligand (e.g., for CTLA-4, CD80 (B7-1) and CD86 (B7-2)), such as a truncated version of one of the ligands. They may be naturally occurring, recombinant or synthetic.

In one embodiment, the subject invention provides a method of identifying a cancer, such as melanoma, e.g., MM, in a subject, the method comprising:
(a) determining the level of an intracellular Gal-3 in:
   i) a test sample obtained from the subject, and
   ii) optionally, a control sample;
(b) optionally, obtaining a reference value corresponding to the level of intracellular Gal-3, or a baseline for said subject; and
(c) identifying the cancer, such as melanoma, e.g., MM, in the subject based on the decreased level of intracellular Gal-3 in the test sample and optionally, administering a therapy to the subject to treat and/or manage the cancer, such as melanoma, e.g., MM.

In some embodiments, the method of identifying a cancer, such as melanoma, e.g., MM, in a subject, may comprise:
(a) determining the level of an extracellular biomarker (e.g., Gal-3) in:
   i) a test sample obtained from the subject, and
   ii) optionally, a control sample;
(b) optionally, obtaining a reference value corresponding to the level of the biomarker (e.g., Gal-3), or a baseline for said subject; and
(c) identifying the cancer, such as melanoma, e.g., MM, in the subject based on the increased level of the biomarker (e.g., Gal-3) in the test sample and optionally, administering a therapy to the subject to treat and/or manage the cancer, such as melanoma, e.g., MM.

In one embodiment, the control sample is obtained from: i) an individual belonging to the same species as the subject and not having the cancer, such as melanoma, e.g., MM, or ii) the subject at a prior time known to be free from the cancer, such as melanoma, e.g., MM.

The term “sample” as used herein refers to any physical sample that includes a cell or a cell extract from a cell, a tissue, a biofluid or an organ including a biopsy sample. The sample can be from a biological source such as a subject, or a portion thereof, or can be from a cell culture. Samples from a biological source can be from a normal or an abnormal organism, such as an organism known to be suffering from a condition or a disease state, or any portion thereof. Samples can also be from any fluid (e.g., blood and serum), tissue or organ including normal and abnormal (diseased) fluid, tissue or organ. Samples from a subject can be used, processed or cultured such that cells from the sample can be sustained in vitro as a primary or continuous cell culture or cell line.

In a specific embodiment, the sample is a skin sample, for example, skin cells, skin extract, and/or skin tissue. Preferably, the skin sample may comprise melanocytes.

The term “subject” or “patient,” as used herein, describes an organism, including mammals such as primates, to which diagnosis, prevention, assessment, and/or treatment according to the present invention can be provided. Mammalian species that can benefit from the disclosed methods of treatment include, but are not limited to, apes, chimpanzees, orangutans, humans, monkeys; domesticated animals such as dogs, cats; live-stocks such as horses, cattle, pigs, sheep, goats, and chickens; and other animals such as mice, rats, guinea pigs, and hamsters.

The terms “treatment” or any grammatical variation thereof (e.g., treat, treating, etc.), as used herein, includes but is not limited to, the application or administration to a subject (or application or administration to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical or mental well-being.

In one embodiment, the subject invention provides a detection method for determining the initiation of a systemic treatment for a cancer, e.g., melanoma, in a subject. The method comprises:
(a) determining the expression level of intracellular Gal-3 in:
   i) a test sample obtained from the subject, and
   ii) optionally, a control sample;
(b) optionally, obtaining a reference value corresponding to the expression level of the intracellular Gal-3, or a baseline for said subject;
(c) determining whether or not to initiate the systemic treatment for the cancer, e.g., melanoma, in the subject based on a reduced expression of the intracellular Gal-3 in the test sample; and
(d) administering a systemic treatment or therapy to the subject to treat and/or manage the cancer, e.g., melanoma.

In one embodiment, the method for determining the initiation of a systemic treatment for a cancer, e.g., melanoma, in a subject may comprise:
(a) determining the level of extracellular Gal-3 in:
   i) a test sample obtained from the subject, and
   ii) optionally, a control sample;
(b) optionally, obtaining a reference value corresponding to the expression level of the extracellular Gal-3, or a baseline for said subject;
(c) determining whether or not to initiate the systemic treatment for the cancer, e.g., melanoma, in the subject based on an increased level of the extracellular Gal-3 in the test sample; and
(d) administering a systemic treatment or therapy to the subject to treat and/or manage the cancer, e.g., melanoma.

In one embodiment, the subject invention provides methods for treating a cancer, e.g., melanoma, preferably, MM, involving the improvement of intracellular Gal-3 expression and/or function. Methods to enhance Gal-3 function or expression would reduce melanoma metastasis progression and/or enhance therapeutic response to conventional therapies, such as immunotherapies, used to treat metastatic melanoma.

In one embodiment, the therapy to the subject to treat cancer, such as melanoma, e.g., MM, may comprise administering to the subject a pharmaceutical composition of the subject invention or a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

A “nucleic acid” according to the invention refers to polynucleotides, such as DNA, RNA, modified DNA, modified RNA (e.g., modRNA) as well as mixtures thereof.

As used herein, “encodes” or “encoding” refers to a DNA and/or RNA sequence that can be processed to generate an RNA and/or polypeptide.

As used herein, “variants” of a protein refer to sequences that have one or more amino acid substitutions, deletions, additions, or insertions. In preferred embodiments, these substitutions, deletions, additions or insertions do not materially adversely affect the protein activity. Variants that retain one or more biological activities are within the scope of the present invention.

“Fragments” and its variants are also within the scope of proteins of the subject invention, so long as the fragment retains one or more biological properties. Preferably, the fragment is at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the full-length protein.

In one embodiment, the subject invention provides a method for treating cancer such as melanoma, e.g., MM, in a subject, the method comprising administering to the subject a pharmaceutical composition according to the subject invention.

In one embodiment, the subject invention provides a method for treating melanoma, preferably, metastatic melanoma, in a subject, the method comprising administering to the subject a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the subject invention provides a method for inhibiting, suppressing, or slowing down melanoma metastasis in a subject, the method comprising administering to the subject a pharmaceutical composition according to the subject invention.

In one embodiment, the subject invention provides a method for inhibiting, suppressing, or slowing down melanoma metastasis in a subject, the method comprising administering to the subject a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the subject has been diagnosed with melanoma.

In one embodiment, the subject invention provides methods for increasing or improving survival of a cancer patient, such as a melanoma patient, preferably, a MM patient, which involve the inhibition of extracellular Gal-3 function and/or the increase of intracellular Gal-3 expression in melanoma cells.

In one embodiment, the method for increasing or improving survival of a melanoma patient, preferably, a MM patient comprises administering to the subject a pharmaceutical composition according to the subject invention.

In one embodiment, the method for increasing or improving survival of a melanoma patient, preferably, a MM patient comprises administering to the subject a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the subject invention provides methods for reducing the expression of pro-metastasis factors, e.g., NFAT1 as well as metastasis-associated proteins in melanoma cells, which involve the overexpression of Gal-3, the method comprising administering to the subject a pharmaceutical composition according to the subject invention.

In one embodiment, the subject invention provides methods for reducing the expression of pro-metastasis factors, e.g., NFAT1 as well as metastasis-associated proteins in melanoma cells, which involve the overexpression of Gal-3, the method comprising administering to the subject a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the subject invention provides methods for reducing the expression of pro-metastasis factors, e.g., NFAT1 as well as metastasis-associated proteins in melanoma cells, which involve the overexpression of Gal-3, the method comprising contacting the melanoma cells with a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof; and/or
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the subject invention provides methods for reducing the expression of pro-metastasis factors, e.g., NFAT1 as well as metastasis-associated proteins in melanoma cells, which involve the overexpression of Gal-3, the method comprising contacting the melanoma cells with a pharmaceutical composition according to the subject invention.

In one embodiment, the subject invention provides a method for slowing the growth of melanoma cells, the method comprising contacting the melanoma cells with a pharmaceutically effective amount of:
1) a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
2) a vector comprising a nucleic acid sequence that encodes Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
3) a cell that overexpresses a nucleic acid sequence of Gal-3, a nucleic acid sequence sharing at least 50%, 55%, 60%, 65%, 70%, 75%, 80%, 85%, 90%, 95%, 96%, 97%, 98% or 99% identity with the nucleic acid sequence that encodes Gal-3, or a nucleic acid sequence that encodes an amino acid sequence sharing at least 50%, 55%, 60%, 65%, 70%, 75%, 80%, 85%, 90%, 95%, 96%, 97%, 98% or 99% identity with Gal-3 protein, biologically-active fragments, variants thereof;
4) a cell that overexpresses an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3, and/or
5) an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3.

In one embodiment, the subject invention provides a method for slowing the growth of melanoma cells, the method comprising contacting the melanoma cells with a pharmaceutical composition according to the subject invention.

Contacting the MM cells with the above 1), 2), 3), 4) and/or 5) or compositions comprises introducing nucleic acids into a cell, either in vitro or in vivo, such methods including, for example, transformation, transduction, transfection, and infection, or Gal-3 protein directly into a cell via, for example, intracellular delivery of the Gal-3 protein. Such introducing results in, for example, the transfection or transduction of the Gal-3 gene into the melanoma cells, which leads to overexpression of Gal-3 in these cells. There are various transfection methods, including physical treatment (e.g., electroporation microinjection, cell squeezing, impalefection, hydrostatic pressure, continuous infusion, sonication, nanoparticles, and magnetofection), chemical materials (e.g., lipofection, and polyplexes) or biological particles (e.g., retrovirus, lentivirus, adenovirus, adeno-associated virus, and herpes simplex virus) that are used as carriers.

The term “vector” refers to a vehicle for introducing a nucleic acid into a cell, which includes, but is not limited to, plasmid, phagemid, virus, bacterium, and vehicle derived from viral or bacterial sources. A “plasmid” is a circular, double-stranded DNA molecule. A useful type of vector for use in the present invention is a viral vector, wherein heterologous DNA sequences are inserted into a viral genome that can be modified to delete one or more viral genes or parts thereof. Certain vectors are capable of autonomous replication in a host cell (e.g., vectors having an origin of replication that functions in the host cell). Other vectors can be stably integrated into the genome of a host cell, and are thereby replicated along with the host genome. In certain embodiments, the vector is a viral vector. Exemplary viral vectors include retroviral, including lentiviral, adenoviral, baculoviral and avian viral vectors.

In one embodiment, the method of treating/preventing/reducing the progression of MM may further comprises administering to the subject one or more therapeutic agents. The therapeutic agent may comprise a chemotherapeutic agent, immunotherapeutic agent, gene therapy or radio therapeutic agent.

The administration routes include, but are not limited to, the local, oral, ophthalmic, nasal, topical, intratumoural, transdermal, intra-articular, parenteral (e.g., intravenous, intraperitoneal, intradermal, subcutaneous or intramuscular), intracranial, intracerebral, intraspinal, intravaginal, intrauterine, or rectal route. Additionally, the composition or therapeutic agents may be administered directly into the tumor of MM.

The term “prevention” or any grammatical variation thereof (e.g., prevent, preventing, etc.), as used herein, includes but is not limited to, at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). The term “prevention” may refer to avoiding, delaying, forestalling, or minimizing one or more unwanted features associated with a disease or disorder, and/or completely or almost completely preventing the development of a disease or disorder and its symptoms altogether. Prevention can further include, but does not require, absolute or complete prevention, meaning the disease or disorder may still develop at a later time and/or with a lesser severity than it would without preventative measures. Prevention can include reducing the severity of the onset of a disease or disorder, and/or inhibiting the progression thereof.

In some embodiments, the therapy to the subject to treat cancer, such as melanoma, e.g., MM, may comprise administering a pharmaceutically effective amount of an amino acid sequence of Gal-3 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with Gal-3 directly to the tumor or cancer cells of the subject.

A further embodiment of the invention provides a method for monitoring the effect of a treatment for a cancer, such as melanoma, e.g., MM, in a subject. A method for monitoring the effect of a treatment for a cancer, such as melanoma, e.g., MM, in a subject may comprise:
(a) determining the level of intracellular Gal-3 in:
   i) a pre-treatment test sample obtained from the subject before the treatment,
   ii) a post-treatment test sample obtained from the subject after the treatment, and
   iii) optionally, a control sample;
(b) optionally obtaining a reference value corresponding to levels of Gal-3, or a baseline for said subject; and
(c) identifying the treatment for the cancer, e.g., MM, in the subject as effective based on an increased level of intracellular Gal-3 in the post-treatment test sample compared to the level of Gal-3 in the pre-treatment test sample and optionally, continuing the treatment in the subject, or
(d) identifying the treatment for the cancer, e.g., MM, in the subject as ineffective if the level of intracellular Gal-3 in the post-treatment test sample remains the same or decreases compared to the level of intracellular Gal-3 in the pre-treatment test sample and optionally, modifying the treatment in the subject.

In certain embodiments, the method for monitoring the effect of a treatment for a cancer, such as melanoma, e.g., MM, in a subject may comprise:
(a) determining the level of extracellular Gal-3 in:
   i) a pre-treatment test sample obtained from the subject before the treatment,
   ii) a post-treatment test sample obtained from the subject after the treatment, and
   iii) optionally, a control sample;
(b) optionally obtaining a reference value corresponding to levels of Gal-3, or a baseline for said subject; and
(c) identifying the treatment for the cancer, e.g., MM, in the subject as effective based on a reduced level of extracellular Gal-3 in the post-treatment test sample compared to the level of Gal-3 in the pre-treatment test sample and optionally, continuing the treatment in the subject, or
(d) identifying the treatment for the cancer, e.g., MM, in the subject as ineffective if the level of extracellular Gal-3 in the post-treatment test sample remains the same or increases compared to the level of extracellular Gal-3 in the pre-treatment test sample and optionally, modifying the treatment in the subject.

In one embodiment, the subject invention provides a method for diagnosing and/or assessing the progression of melanoma, preferably, MM in a subject, the method comprising:
(i) assessing the expression level of intracellular Gal-3 in a sample obtained from the subject;
(ii) comparing the expression level of intracellular Gal-3 in the sample to a reference derived from the expression level of intracellular Gal-3 in a sample obtained from a healthy subject, or a baseline for said subject; and
(iii) determining the progression of melanoma, preferably, MM in the subject based on whether the expression level of intracellular Gal-3 in the subject is up-regulated or down-regulated.

In further embodiments, if intracellular Gal-3 is up-regulated in the sample, it is indicative of an improvement in the subject's condition whereas if intracellular Gal-3 level remains the same or down-regulated in the sample, it is indicative that the subject's condition is worsened, and the melanoma may be progressing to the metastatic stage.

In one embodiment, the method for diagnosing and/or assessing the progression of melanoma, preferably, MM in a subject, may comprise:
(i) assessing the level of extracellular Gal-3 in a sample obtained from the subject;
(ii) comparing the level of extracellular Gal-3 in the sample to a reference derived from the level of extracellular Gal-3 in a sample obtained from a healthy subject, or a baseline for said subject; and
(iii) determining the progression of melanoma, preferably, MM in the subject based on whether the level of extracellular Gal-3 in the subject is up-regulated or down-regulated.

In further embodiments, if extracellular Gal-3 is down-regulated in the sample, it is indicative of an improvement in the subject's condition whereas if intracellular Gal-3 level remains the same or up-regulated in the sample, it is indicative that the subject's condition is worsened and the melanoma may be progressing to the metastatic stage.

In one embodiment, the subject invention provides a method for stratifying a tumor stage (e.g., of MM) in a subject, the method comprising:
(i) assessing the expression level of intracellular Gal-3 in a tumor sample obtained from the subject;
(ii) comparing the expression level of intracellular Gal-3 in the tumor sample to a reference derived from the expression level of intracellular Gal-3 in healthy samples obtained from healthy subjects, or a baseline for said subject; and
(iii) determining the tumor stage in the subject based on whether the expression level of intracellular Gal-3 in the subject is up-regulated or down-regulated.

In one embodiment, the subject invention provides a method for predicting an outcome of an anti-cancer therapy, in a subject, the method comprising:
(i) assessing the expression level of i intracellular Gal-3 obtained from the subject;
(ii) comparing the expression level of intracellular Gal-3 in the sample to a reference derived from the expression level of intracellular Gal-3 in a sample obtained from healthy subjects, or a baseline for said subject; and
(iii) determining/predicting the outcome of the anti-cancer therapy by assessing whether the level of intracellular Gal-3 in the subject is up-regulated or down-regulated, wherein a up-regulation in the expression of intracellular Gal-3 indicates that the cancer, e.g., MM, will be responsive and/or sensitive to the anti-cancer therapy.

In one embodiment, the subject invention provides a method for assessing the response of a melanoma subject to an anti-melanoma therapy, the method comprising:
assessing an expression level of intracellular Gal-3 in a sample obtained from the melanoma subject before and after the anti-melanoma therapy;
comparing the expression level of intracellular Gal-3 in the sample before and after the anti-melanoma therapy; and
determining the melanoma subject being responsive to the anti-melanoma therapy by a increased expression level of intracellular Gal-3 in the sample, or determining the melanoma subject being non-responsive to the anti-melanoma therapy if the expression level of intracellular Gal-3 remains the same or decreases.

In a specific embodiment, the subject may have hypoxia. In a specific embodiment, the subject may not have hypoxia. In a specific embodiment, the MM cells are under hypoxia. In a specific embodiment, the MM cells are not under hypoxia.

In a further embodiment, the melanoma or MM may be a drug-resistant melanoma or MM. In a preferred embodiment, the melanoma is an ICI therapy-resistant melanoma.

In one embodiment, the subject invention provides a method of identifying melanoma being MM in a subject, wherein the subject has been diagnosed with melanoma, the method comprising:
(a) determining the level of an intracellular Gal-3 in:
   i) a test sample obtained from the subject, and
   ii) optionally, a control sample;
(b) optionally, obtaining a reference value corresponding to the level of intracellular Gal-3, or a baseline for said subject; and
(c) identifying the melanoma being MM based on the decreased level of intracellular Gal-3 in the test sample and optionally, administering a therapy to the subject to treat and/or manage MM.

In a further embodiment, a decreased level of intracellular Gal-3 in the test sample compared to the reference is indicative that the melanoma of the subject is metastatic melanoma whereas in the absence of such decreased level of intracellular Gal-3 in the test sample compared to the reference is indicative that the melanoma of the subject is not metastatic melanoma.

In a specific embodiment, the reference can be an expression level of intracellular Gal-3 from a healthy subject, a subject with primary melanoma, a subject with circulating melanoma or a subject having been diagnosed with a melanoma that is not metastatic.

In one embodiment, the method of the subject invention further comprises administering to the subject a composition comprising an inhibitor that inhibits the level of extracellular Gal-3, the binding of extracellular Gal-3 to its ligand, and/or the secretion of Gal-3 outside of the cells.

In one embodiment, methods of the subject invention use a combination of therapies that 1) increases the expression level of intracellular Gal-3 and 2) reduce the level of extracellular Gal-3 and/or inhibits the function of extracellular Gal-3 by, for example, inhibiting the binding of extracellular Gal-3 to its ligands. Such combined therapy provides a synergistic effect in, for example, treating melanoma, e.g., MM, suppressing melanoma metastasis, sensitizing a patient to immunotherapies, and improving survival of a patient.

In one embodiment, the melanoma cells have reduced expression level of GCNT2, for example, prior to contacting the composition of the subject invention.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

When ranges are used herein, such as for dose ranges, combinations and subcombinations of ranges (e.g., subranges within the disclosed range), specific embodiments therein are intended to be explicitly included.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### EXAMPLES

### Methods

### Gene Expression Data Collection and Processing.

Transcriptomic data of 33 cancers, including skin cutaneous melanoma (SKCM) samples (n=481), were retrieved from The Cancer Genome Atlas (TCGA) database, while normal skin samples (n=977) were collected from Genotype Tissue Expression (GTEx) database. Data were analyzed and visualized using UCSC Xena (xena.ucsc.edu). Exclusion criteria were as follows: (1) Samples with missing data regarding sample type or Gal-3 expression; (2) TCGA melanoma samples categorized as solid tissue normal or additional metastatic. As a result, a total of 10,284 samples from 33 cancer types, including 471 SKCM samples (103 primary melanoma and 368 metastatic melanoma) and 812 normal skin samples were included in the study. The obtained gene expression profiles were generated using the Illumina HiSeq 2000 RNA Sequencing platform and presented as log 2(value+1) transformed RSEM normalized count. The Gene Expression Omnibus (GEO) browser (www.ncbi.nlm.nih.gov/geo/browse/) was searched for datasets that include primary and metastatic melanoma samples. The microarray dataset GSE8401 (included 31 primary melanoma samples and 52 metastatic melanoma samples) was uploaded to the interactive web tool (GEO2R) (www.ncbi.nlm.nih.gov/gco/geo2r/) that uses ‘limma’ package of R programming language for gene expression analysis. The RNA-seq dataset GSE157740 (included 2 melanoma CTC lines and their patient-matched primary or metastatic specimens) was analyzed by the online GREIN platform (www.ilincs.org/apps/grein/?gse=). An adjusted p value <0.05 was considered significant. The GEO browser was also searched for single-cell RNA sequencing (scRNA-seq) data of immune cells from human melanoma tumors. The scRNA-seq datasets GSE139249 (included 5 samples of melanoma metastases) and GSE123139 (included primary and metastatic melanoma samples from 25 patients) were analyzed and visualized using the Tumor Immune Single-cell Hub 2 (TISCH2) tool to determine Gal-3 expression patterns across major cell lineages in each scRNA-seq cohort. The TISCH2 platform (tisch.comp-genomics.org/home/) provides the protocol of data collection, processing, and cell-type annotation.

### Patients and Sample Processing.

A total of 44 melanoma patients (AJCC Stage 0 n=9, Stage I n=7, Stage II n=9, Stage III n=8, Stage IV n=11) who presented to New York University (NYU) Langone Medical Center were included in this study. Blood samples were collected from these patients at the time of primary diagnosis and/or recurrence. All patients had histologically confirmed melanoma. Blood samples from healthy control donors were obtained from the Biospecimen Repository Facility at Miami Cancer Institute Baptist Health-South Florida. To minimize pre-analytical variability, all samples were routinely collected, processed, and stored using standardized protocols. For consistency and reproducibility, samples were processed within 90 min after collection by centrifugation at 2500 rpm for 10 min at room temperature. Aliquots (1 mL) of sera were stored in cryovials at −80° C. until further analysis.

### ELISA of Gal-3 in Melanoma Patient Sera.

Blood samples of 44 melanoma patients were obtained from New York University (NYU) Langone Medical Center. Gal-3 was measured in the serum samples using a commercial enzyme-linked immunosorbent assay (ELISA) Kit (DGAL30, R & D systems, Minneapolis, MN, USA), according to the manufacturer's instructions. The absorbance of samples was measured at 450 nm and a standard curve was generated using a Cytation 5 reader and Gen5 software (BioTek Instruments, Winooski, VT, USA). All samples were analyzed in triplicates. Gal-3 levels in melanoma patients were compared with those in age-matched healthy control donors. The results were expressed as ng/ml. Reagents details are available in Table 1.

TABLE 1
Antibodies and reagents used for flow cytometry, western blotting, RT-PCR, and ELISA

| Antibodies/Reagents | Source | Identifier | Concentrations |
|---|---|---|---|
| Human Galectin-3 Quantikine | R&D | Cat# 926-32210 | N/A |
| ELISA Kit | Systems ™ | | |
| IRDye ® 800CW Goat anti-Rabbit | LI-COR | Cat# DGAL30 | 1:10000 |
| IgG (H + L) | Biosciences | | (Western) |
| IRDye ® 800CW Goat anti-Mouse | LI-COR | Cat# 926-32210 | 1:10000 |
| IgG (H + L) | Biosciences | | (Western) |
| IRDye ® 800CW Goat anti-Rat IgG | LI-COR | Cat# 926-32219 | 1:10000 |
| (H + L) | Biosciences | | (Western) |
| IRDye ® 680LT Donkey anti-Rabbit | LI-COR | Cat# 926-68023 | 1:10000 |
| IgG (H + L) | Biosciences | | (Western) |
| NFAT1 (D43B1) XP ® Rabbit mAb | Cell Signaling | Cat# 4370 | 1:1000 |
| | | | (Western) |
| Phospho-p44/42 MAPK (Erk1/2) | Cell Signaling | Cat# 5863 | 1:1000 |
| (Thr202/Tyr204) (D13.14.4E) XP ® | | | (Western) |
| Rabbit mAb | | | |
| ERK1/ERK2 Mouse anti-Human, | R&D Systems ™ | Cat# MAB1576 | 1:1000 |
| Mouse, Rat, Clone: 216703 | TM | | (Western) |
| Phospho-Akt (Ser473) (D9E) XP ® | Cell Signaling | Cat# 4060S | 1:1000 |
| Rabbit mAb #4060 | | | (Western) |
| Akt (pan) (40D4) Mouse mAb | Cell Signaling | Cat# 2920S | 1:1000 |
| | | | (Western) |
| Rabbit Beta actin (D6A8) | Cell Signaling | Cat# 8457S | 1:1000 |
| | | | (Western) |
| Anti-beta Actin, Clone: mAbcam | AbcamTM | Cat# ab8226 | 1:1000 |
| 8226 | | | (Western) |
| Purified anti-mouse/human Mac-2 | Biolegend | Cat# 125402 | 1:1000 |
| (Galectin-3) Antibody | | | (Western) |
| Alexa Fluor ® 647 antimouse/ | Biolegend | Cat# 125408 | 1 μg/ml |
| human Mac-2 (Galectin-3) | | | (Flow |
| Antibody | | | cytometry) |
| Alexa Fluor ® 647 Rat IgG2a, κ | Biolegend | Cat# 400526 | 1 μg/ml |
| Isotype Ctrl Antibody | | | (Flow |
| | | | cytometry) |
| LIVE/DEAD ™ Fixable Aqua Dead | Fisher | Cat# 50-112-1525 | 1:1000 |
| Cell Stain Kit, for 600 nm excitation | | | (Flow |
| | | | cytometry) |
| Human LGALS3 TaqMan ® primer | Thermo | AssayID: | N/A |
| | Scientific | Hs00173587_m1 | |
| Mouse LGALS3 TaqMan ® primer | Thermo | AssayID: | N/A |
| | Scientific | Mm00802901_m1 | |
| Human GAPDH TaqMan ® primer | Thermo | AssayID: | N/A |
| | Scientific | Hs02786624_g1 | |
| Mouse GAPDH TaqMan ® primer | Thermo | AssayID: | N/A |
| | Scientific | Mm99999915_g1 | |
| Human NFAT1 TaqMan ® primer | Thermo | AssayID: | N/A |
| | Scientific | Hs00905451_m1 | |
| Mouse NFAT1 TaqMan ® primer | Thermo | AssayID: | N/A |
| | Scientific | Mm01240677_m1 | |
| RNeasy Plus Mini Kit (250) | QIAGEN | Cat# 74136 | N/A |
| TaqMan ® Fast Advanced Master | Thermo | Cat# 4444557 | N/A |
| Mix | Scientific | | |
| SuperScript ™ VILO ™ CDNA | Invitrogen ™ | Cat# 11-754-050 | N/A |
| Synthesis Kit | | | |
| RIPA Lysis and Extraction Buffer | Thermo | Cat# 89900 | N/A |
| | Scientific | | |
| Halt ™ Protease and Phosphatase | Thermo | Cat#78440 | N/A |
| Inhibitor Single-Use Cocktail (100X) | Scientific | | |
| BCA protein assay kit | Thermo | Cat# PI23227 | N/A |
| | Scientific | | |
| XT Sample Buffer, 4X | BioRad | Cat#1610791 | 1X |
| Immobilon PVDF | Millipore | Cat# IPFL00010 | N/A |
| Criterion 4-12% Bis-Tris Precast | BioRad | Cat# 3450123 | N/A |
| Gels-12 wells + 2 | | | |
| Intercept ® (TBS) blocking buffer | LI-COR | Cat# J19943.K2 | N/A |
| | Biosciences | | |
| Paraformaldehyde Solution | Thermo | Cat# NC1660551 | 4% in PBS |
| | Scientific | | |
| Crystal violet | Alfa Aesar | Cat# B21932 | N/A |
| Bright-Glo ™ Luciferase Assay | Promega | Cat# E2610 | N/A |
| System | | | |
| VivoGlo ™ Luciferin, In Vivo Grade | Promega | Cat# P1042 | N/A |
| Firefly Luciferase Lentivirus | BPS | Cat# 79902-P | N/A |
| (Puromycin) | Bioscience | | |
| Expression Negative Control | BPS | Cat# 79692-P | N/A |
| Lentivirus (Puromycin) | Bioscience | | |
| Accutase-Enzyme Cell Detachment | Fisher | Cat# 50-112-9055 | N/A |
| Medium | | | |

### Cell Lines and Cell Culture.

Human melanoma cell lines (SK-MEL-2, SK-MEL-5, G361, UACC62 and A375), and B16F0 murine melanoma cell line were purchased from the American Type Culture Collection (ATCC, Manassas, VA, USA). B16/BL6 murine melanoma cell line was purchased from Accegen Biotechnology (Fairfield, NJ, USA). Cells were grown in their respective culture media (Dulbecco's modified Eagle's medium (DMEM) for G361, UACC62, SK-MEL-5, A375 and B16F0 cell lines, and RPMI 1640 medium for SK-MEL-2 and B16/BL6 cell lines) supplemented with 10% fetal bovine serum (FBS) (Atlanta Biologicals, Flowery Branch, GA, USA) and 1% Antibiotic-Anti-mitotic (Gibco, Waltham, MA, USA), and maintained in a humidified 37° C. incubator with 5% CO2. Cell lines were passaged when they reached approximately 80% confluency and were regularly tested with PlasmoTest (InvivoGen, San Diego, CA, USA) to ensure the absence of mycoplasma contamination. Reagents details are available in Table 1.

### Clonogenic Assay.

Cells were seeded in a 6-well culture plate and incubated at 37° C. Fresh medium was replaced every after 24-48 h until the 10 days of incubation. Colony formation was evaluated by fixing and staining the cells after 10 days of incubation. Colony formation was evaluated by fixing the cells with 100% methanol, staining with 0.5% crystal violet, and imaging using EVOS® FL Imaging System (Life Technologies, Grand Island, NY, USA). Cell survival was calculated (treated count/untreated count). Reagents details are available in Table 1.

### Transwell Migration and Invasion Assays.

Cells were seeded on top of the filter membrane in an 8.0-μm-pore transwell insert (Corning Incorporated, Corning, NY, USA) with serum-free medium. The inserts were placed into a 24-well plate with medium supplemented with 30% FBS as a chemo-attractant. For invasion assay, the top filter membrane was coated with a layer of Matrigel before loading the cells. After incubation for 48 h, cells in the upper chamber were removed by a cotton swab gently. The migrated/invaded cells attached to the lower surface of the filters were fixed in 4% paraformaldehyde and stained with 0.5% crystal violet. For quantification, the migrated/invaded cells were counted at 5 randomly selected areas in each well under 40× magnification using EVOS® FL Imaging System (Life Technologies, Grand Island, NY, USA). Data were expressed as mean±SD from three independent experiments. Reagents details are available in Table 1.

### RT-qPCR Analysis.

For gene expression analysis, RNA was extracted using RNeasy Plus kit (mini) (Qiagen, Ontario, Canada) and cDNA synthesized using SuperScript™ VILO™ cDNA synthesis kit (Invitrogen; Thermo Fisher, CA, USA) per manufacturer's protocol. Real-time quantitative PCR was then performed with TaqMan® Fast Advanced Master Mix (Applied Biosystems, Foster City, CA, USA) and TaqMan® primers to amplify genes (LGALS3, NFAT1, and GAPDH as an internal control) per manufacturer's protocol. Reagents details are available in Table 1.

### Immunoblotting.

Cells were lysed in Pierce™ RIPA buffer (Thermo Scientific™) with protease and phosphatase inhibitor cocktail (Thermo Scientific™). After a 30 min incubation on ice, cell lysates were centrifuged for 10 mins at 10,000 RPM in 4° C. Protein concentrations were calculated using Pierce™ BCA protein assay kit (Thermo Scientific™) per manufacturer protocol, and equal protein amounts from each sample were prepared in Laemmli sample buffer (Bio-Rad, Hercules, CA, USA). Samples were boiled for 5 mins and subsequently loaded on a 4-12% gradient SDS PAGE gel (BioRad) for electrophoresis. Separated proteins were transferred to polyvinylidene fluoride (PVDF) membranes (Millipore, Burlington, Massachusetts), blocked for 1 h at room temperature with Intercept® (TBS) blocking buffer (LI-COR, Lincoln, Nebraska, USA), and incubated overnight at 4° C. with primary antibodies against Gal-3, NFAT1, p-AKT, total AKT, p-ERK1/2, total ERK1/2, and □-actin proteins. Membranes were later washed and incubated with IRDye® secondary antibodies for 1 hr at room temperature. Membranes were then visualized and analyzed with Li-Cor imager. Reagents details are available in Table 1.

### Flow Cytometry.

To analyze surface expression of Gal-3, cells were harvested using Accutase (Fisher), washed with PBS and resuspended in Alexa Fluor 647 anti-mouse/human Mac-2 (Galectin-3) antibody (Biolegend) and Aqua Live/Dead stain for 30 min on ice. Single-color, isotype, and unstained controls were also prepared for validation. Cells were washed, resuspended in 200 μl of PBS. Flow cytometric were acquired using FACSCelesta (BD Biosciences, San Jose, CA, USA) and analyzed using the FlowJo software (Tree Star, Ashland, OR, USA). Reagents details are available in Table 1.

### Generation of Stable Gal-3 Silencing.

SK-MEL-2 and G361 cells were infected with three different shRNA-carrying lentiviral particles directed against Gal-3 or with scrambled control shRNA-carrying lentiviral particles (GeneCopoeia, Rockville, MD, USA) according to the manufacturer's protocol. Briefly, 2×104 cells/well were plated in 96-well plate and incubated overnight with the lentiviral particles at a multiplicity of infection (MOI) of 5 in serum-free medium. On the next day, cells were replaced with fresh complete medium, and two days later, cells were selected in 200 μg/mL Hygromycin (Corning, Corning, NY, USA) containing medium for 1 week to generate stable lines (ScrCtrl and Gal3KD). Reagents details are available in Table 1. Oligonucleotide details are available in Table 2.

TABLE 2
Oligonucleotide targets for each LGALS3 shRNA lentiviral particle

| Gene | Oligonucleotide sequences | Comments |
|---|---|---|
| Human LGALS3 shRNA #1 | CCTCGCATGCTGATAACAATT | shRNA target sequence |
| | (SEQ ID NO: 1) | |
| Human LGALS3 shRNA #2 | CCACGCTTCAATGAGAACAAC | shRNA target sequence |
| | (SEQ ID NO: 2) | |
| Human LGALS3 shRNA #3 | GGAATTTCTGGTGACATAGAC | shRNA target sequence |
| | (SEQ ID NO: 3) | |
| Human LGALS3 scrambled | GCTTCGCGCCGTAGTCTTA | Non-target sequence |
| control | (SEQ ID NO: 4) | |

### Generation of Stable Gal-3 Overexpression.

A375 cells were infected with Lentiviral particles for LGALS3, while empty vectors were used as a control (GeneCopoeia, Rockville, MD, USA) according to the manufacturer's protocol. Briefly, 2× 10⁴ cells/well were plated in 96-well plate and incubated overnight with the lentiviral particles at a multiplicity of infection (MOI) of 5 in serum-free medium. On the next day, cells were replaced with fresh complete medium, and two days later, cells were selected in 4 μg/mL puromycin (Gibco, Waltham, MA, USA) containing medium for 1 week to generate stable lines (EVCtrl and Gal3OE). Reagents details are available in Table 1. Oligonucleotide details are available in Table 2.

### Generation of Luciferase-Expressing SK-MEL-2 Cells.

SK-MEL-2 and G361 ScrCtrl and Gal3KD cells were transduced with firefly luciferase lentivirus or negative control lentivirus (BPS Bioscience, San Diego, CA, USA) according to the manufacturer's protocol. Briefly, 2×10⁴ of either ScrCtrl or Gal3KD cells were plated in 96-well plate and incubated overnight with the lentiviral particles at a multiplicity of infection (MOI) of 5 in serum-free medium. On the next day, lentivirus-containing medium was replaced with fresh complete medium, and two days later, cells were selected for 10 days in selection medium containing 4 μg/mL puromycin (Gibco, Waltham, MA, USA) to generate stable lines expressing luciferase cells (ScrCtrl.Luc and Gal3KD.Luc) and negative control cells (Scr.NegCtrl and Gal3KD.NegCtrl) respectively. An in vitro luciferase activity assay was performed to verify successful expression of firefly luciferase using the Bright-Glo™ Luciferase Assay System (Promega, Madison, WI, USA) per manufacturer protocol. Briefly, luciferase-expressing and negative control ScrCtrl and Gal3KD cells were seeded in a 96-well white bottomed plate in a serial dilution of 250,000, 125,000, 62,500, and 31,250 cells/well. On the next day, the Bright-Glo Luciferase Assay reagent was added to each well and incubated for 5 min. The number of photons emitted per second over a 30 sec exposure period was measured using the AMI HT imager (Spectral Instruments Imaging, Tucson, AZ), and quantified using the Aura Imaging Software (spectralinvivo.com/software/).

### Tumor Xenograft Mouse Model.

A total of 1×10⁶ ScrCtrl or Gal3KD cells were injected subcutaneously into the right flank of 6- to 8-week-old NOD-SCID IL-2Rγ-deficient (NSG) mice (bred in-house, strain from the Jackson Laboratory). Both male and female mice were equally included in the studies. Tumor growth was assessed every 1-2 weeks by a vernier caliper. Tumor volume was calculated using the formula: V=LW²/2, where L is the length (longest dimension), and W is the width (shortest dimension). At the endpoint of the experiment, the mice were sacrificed, and tumors were excised and imaged. All experimental procedures were conducted as per FIU IACUC protocol. Reagents details are available in Table 1.

### In Vivo Metastasis Assay.

1×10⁶ cells of either ScrCtrl. Luc or Gal3KD.Luc cells were injected intravenously into the tail veins of 6- to 8-week-old NOD-SCID IL-2Rγ-deficient (NSG) mice (bred in-house, strain from the Jackson Laboratory). Metastatic tumor formation and colonization were monitored bi-weekly using an AMI HT imager. Briefly, mice were anesthetized with 2% isoflurane and maintained under anesthesia by continuous inhalation of isoflurane until imaging was complete. Mice were injected intraperitoneally with 200 μl of 15 mg/ml D-luciferin solution (VivoGlo™Luciferin, In Vivo Grade, Promega, Madison, WI, USA). Ventral images of the mice were taken 10 min later using AMI HT imaging system and quantified using the Aura Imaging Software. The conditions for bioluminescence acquisition were as follows: open emission filter, exposure time 30 seconds, binning medium for 8, field of view 12.9 cm, and f/stop as 1. Rainbow images show the relative levels of luminescence ranging from low (blue), to medium (green), to high (yellow/red). Reagents details are available in Table 1.

### Statistical Analysis.

Prism 8.0 software (GraphPad) was used for statistical analysis. For normally distributed data involving two groups, unpaired two-tailed Student's t-test was used. For non-normally distributed data, analysis was performed using a Mann-Whitney test; normality was assessed using a Shapiro-Wilk test. Spearman's correlation test was used to investigate the correlation between the expression levels of Gal-3 and NFAT1 in TCGA melanoma data. Throughout, data are presented as the means±SEM, unless otherwise noted. P value of <0.05 was considered statistically significant.

### Data Availability

The data analyzed in this study were obtained from The Cancer Genome Atlas (TCGA) database, which is publicly available through the Genomic Data Commons (GDC) Data Portal (portal.gdc.cancer.gov/) and the Gene Expression Omnibus (GEO) database at GSE8401, GSE157740, GSE139249, and GSE123139.

### Example 1—Gal-3 is Downregulated in Metastatic Melanomas Compared to Primary Melanomas

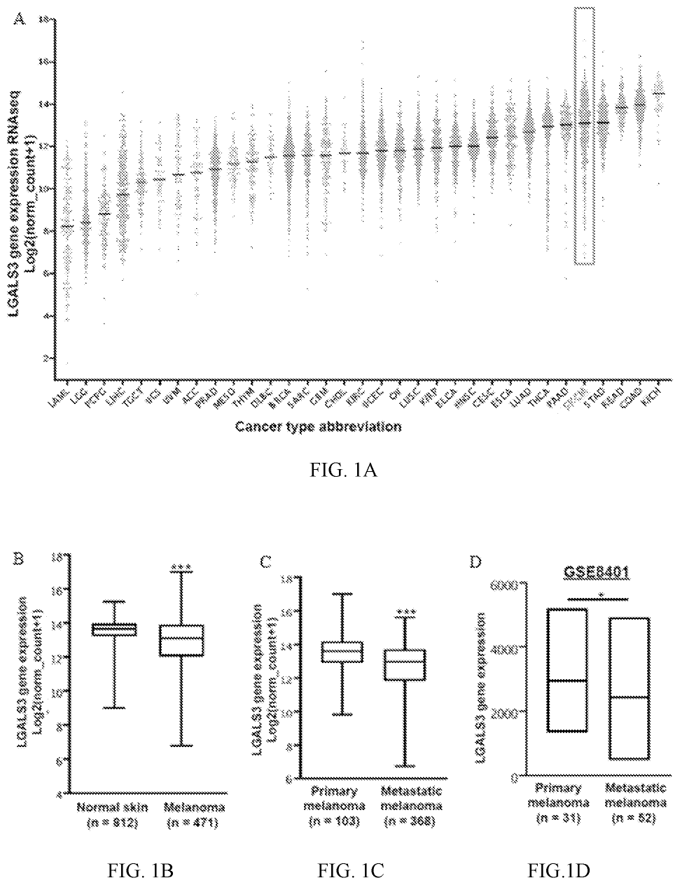

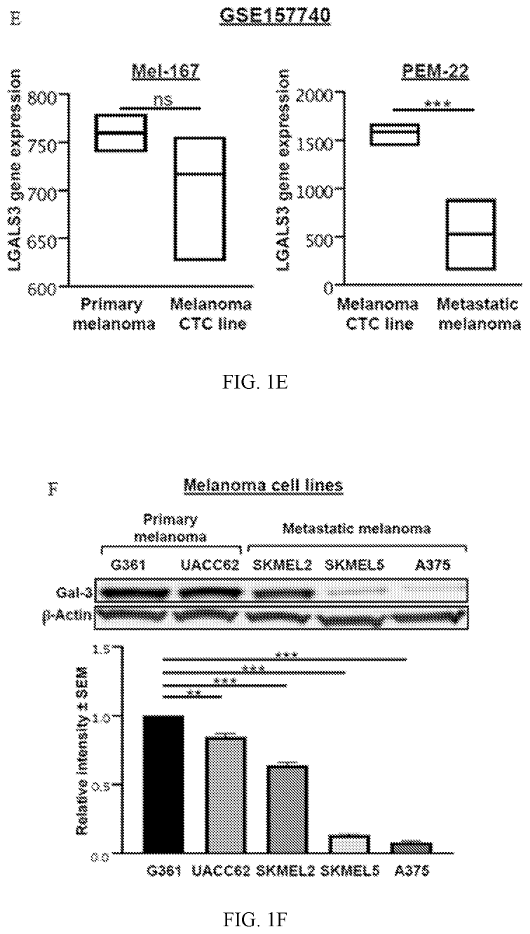

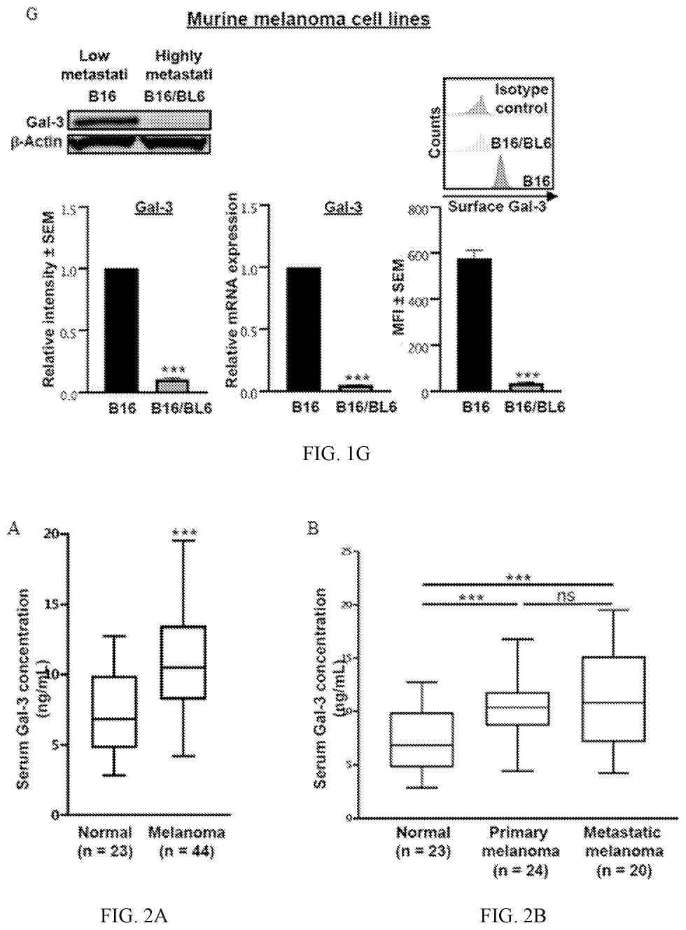

To explore Gal-3 expression in melanomas, a pan-cancer analysis of Gal-3 expression was first applied in samples from 33 different types of cancer (n=10,284) collected in the TCGA database. It is found that melanoma is among the top Gal-3-expressing tumors (FIG. 1A). However, Gal-3 expression was significantly downregulated in the TCGA melanoma samples (n=471) compared to its expression in normal skin tissues (n=812) (p<0.001) (FIG. 1B).

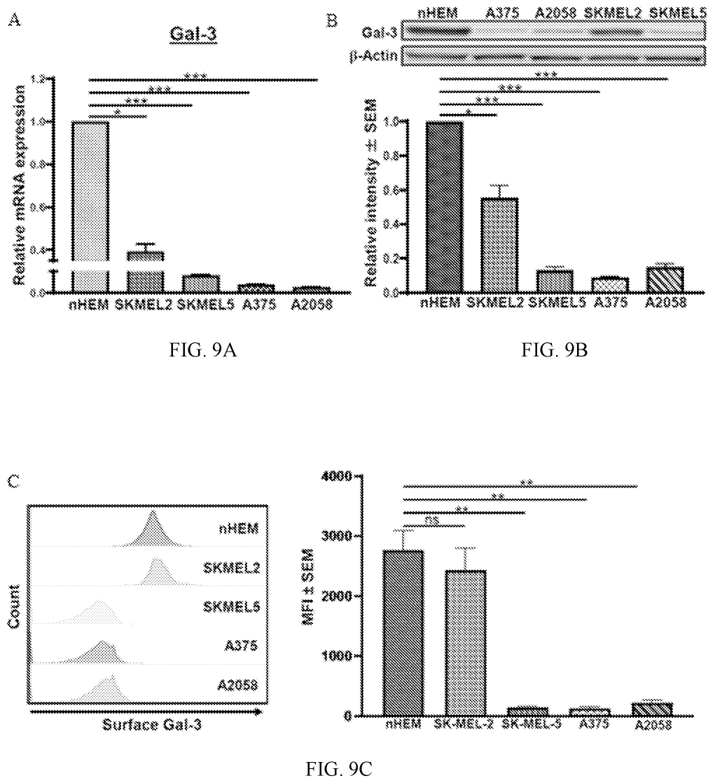

Considering that melanocytes are underrepresented in the normal skin epidermal layer with a keratinocyte-melanocyte ratio of approximately 35:1, Gal-3 expression was then assessed in normal human epidermal melanocytes (nHEM) versus a panel of human melanoma cell lines (A375, A2058, SK-MEL-2 and SK-Mel-5) using RT-qPCR (FIG. 9A) and immunoblotting (FIG. 9B). The results showed that nHEM exhibited significantly higher Gal-3 expression compared with the human melanoma cell lines SK-MEL-2 (p<0.05), SK-MEL-5 (p<0.001), A375 (p<0.001), and A2058 (p<0.001). The expression of membrane bound Gal-3 was also analyzed by flow cytometry (FIG. 9C), which showed a significantly higher expression of Gal-3 on nHEM surface compared to SK-MEL-5 (p<0.01), A375 (p<0.01), and A2058 (p<0.01), with no significant difference of surface expression of Gal-3 between nHEM and SK-MEL-2. These data indicate that melanoma cells express lower though variable levels of intracellular and extracellular Gal-3 compared with normal melanocytes.

Gal-3 expression profiles of primary melanoma samples (n=103) versus metastatic melanoma samples (n=368) were then investigated using the TCGA database. Data analysis revealed a significantly higher expression of Gal-3 in primary melanomas compared with metastatic melanomas (p<0.001) (FIG. 1C). Consistent with this assessment, analysis of the microarray dataset GSE8401 demonstrated lower expression of Gal-3 in metastatic melanoma samples (n=52) compared to primary melanomas (n=31) (p<0.05) (FIG. 1D).

Furthermore, the RNA-seq dataset GSE157740 was analyzed to compare Gal-3 transcriptional profiles of PEM-22 melanoma CTC lines versus 6 patient-matched-independent archival metastatic lesions and to compare transcriptional profiles of Mel-167 CTCs versus patient-matched primary tumor. Data analysis demonstrated that Gal-3 expression in Mel-167 CTCs was lower than its levels in patient-matched primary tumors (FIG. 1E, left panel). On the other hand, Gal-3 expression is significantly downregulated in metastatic lesions compared to patient-matched PEM-22 CTCs (p<0.001), suggesting a predilection of Gal-3-deficient cells in metastatic foci (FIG. 1E, right panel).

To further confirm the negative correlation between the metastatic potential of melanoma cells and cell-intrinsic Gal-3 expression levels, Gal-3 expression was assessed in a panel of human primary (G361 and UACC62), poorly metastatic (SK-MEL-2), and highly metastatic (SK-MEL-5 and A375) melanoma cell lines using immunoblotting (FIG. 1F). The results showed that human melanoma cells express variable levels of Gal-3 with higher tendency of Gal-3 loss within the highly metastatic melanoma cell lines. Consistently, a marked decline of Gal-3 expression was observed in the highly metastatic B16/BL6 murine melanoma cell line versus the parent low metastatic B16 cell line using immunoblotting, RT-qPCR, and flow cytometry analysis (FIG. 1G) (p<0.001, p<0.001, p<0.001, respectively). Together, these results suggest that loss of intracellular Gal-3 in melanoma cells corresponds with higher metastatic potential.

### Example 2—Gal-3 is Elevated in the Sera of Melanoma Patients Though does not Correlate to Tumor Progression

To explore the possible association of serum Gal-3 levels with melanoma progression, circulating Gal-3 levels were measured in sera from 44 patients with melanoma and 23 age matched healthy controls (Table 3).

TABLE 3
Demographics and clinical features of patients with
melanoma and healthy controls

| | Patients with | Healthy |
|---|---|---|
| | melanoma | Controls |
| | (n = 44) | (n = 23) |
| Sex, n (%) | | |
| Male | 26 (72.2) | 10 (27.8) |
| Female | 18 (58.1) | 13 (41.9) |
| Age at diagnosis, y (mean ± SD) | 62.8 ± 13.1 | 53.0 ± 14.2 |
| Melanoma stage, n (%) | | |
| Primary melanoma | 25 (56.8) | |
| Stage 0 | 9 (20.5) | — |
| Stage I | 7 (15.9) | |
| Stage II | 9 (20.5) | |
| Metastatic melanoma | 19 (43.2) | |
| Stage III | 8 (18.2) | |
| Stage IV | 11 (25) | |
| Metastatic site, n (%) | (n = 19) | |
| Lymph nodes | 7 (36.8) | |
| In-transit | 4 (21.1) | |
| Systemic | | |
| Lung | 7 (36.8) | |
| Liver | 6 (31.6) | — |
| Bone | 2 (10.5) | |
| Skin | 2 (10.5) | |
| Brain | 1 (5.3) | |
| Pancreas | 1 (5.3) | |
| Spleen | 1 (5.3) | |

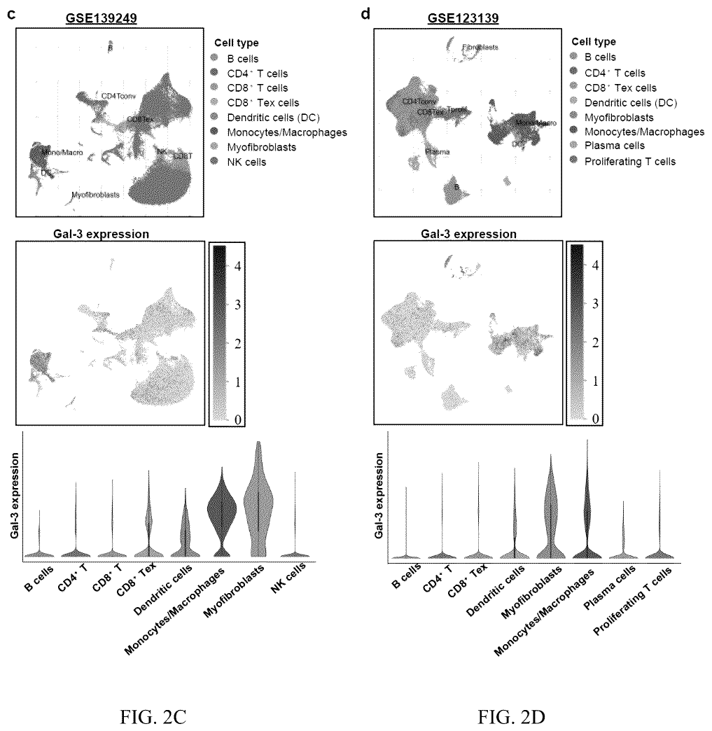

Twenty-five patients presented with primary melanoma (stage 0, I or II), while 19 patients presented with lymph node (LN) and/or visceral melanoma metastasis (stage III/IV). The results show that melanoma patients had significantly elevated circulating levels of Gal-3 (10.8±3.5 ng/mL) compared with age-matched healthy subjects (7.2±3.0 ng/ml) (p<0.001) (FIG. 2A). No significant difference of circulating Gal-3 levels was detected between patients who developed metastases, including regional LN metastases, and those with non-metastatic melanoma (11.3±4.2 ng/mL vs. 10.4±2.7 ng/ml, p=0.41) (FIG. 2B). These data indicate that melanomas are associated with elevated serum Gal-3 with lack of correlation between its levels and melanoma stage.

### Example 3—Gal-3 is Expressed by Other Non-Melanoma Cells in the Tumor Microenvironment (TME)

To determine possible sources for extracellular and circulating Gal-3 distinct from melanoma cells, scRNA-seq data of immune and stromal cells within human melanoma samples were analyzed. Data analysis of two GEO datasets, GSE139249 (FIG. 2C) and GSE123139 (FIG. 2D), using the TISCH2 webtool revealed variability in Gal-3 expression among immune and stromal cells in the TME, with monocytes/macrophages and myofibroblasts constituting the major cellular contributors to the Gal-3 pool in the melanoma microenvironment. These findings indicate that non-melanoma immune and stromal cells within the TME likely represent fundamental sources of extracellular and circulating Gal-3 in melanoma patients.

### Example 4—Ablating Gal-3 in Melanoma Cells Enhances the Metastatic Behavior

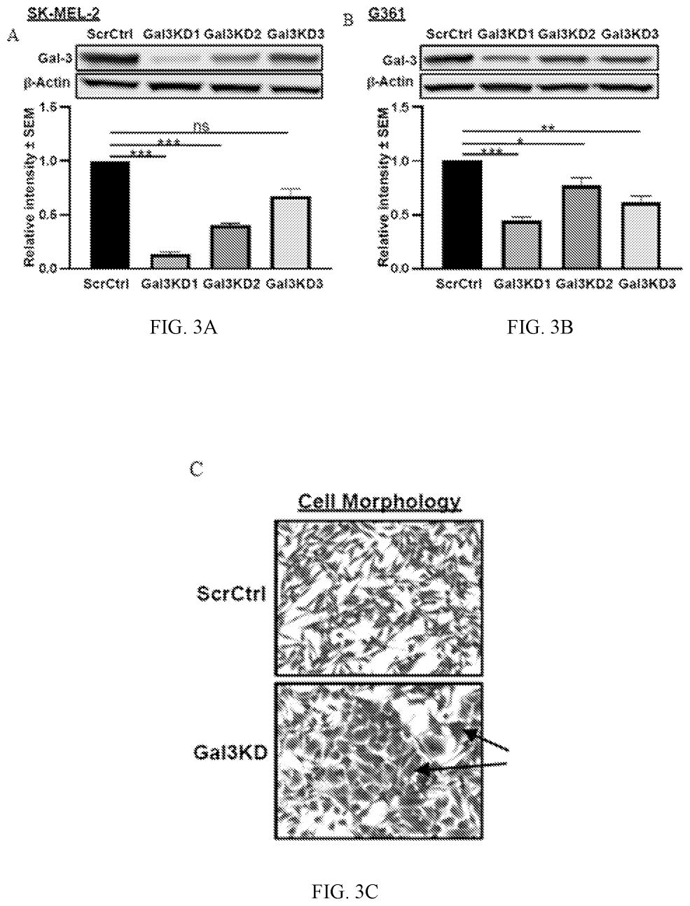

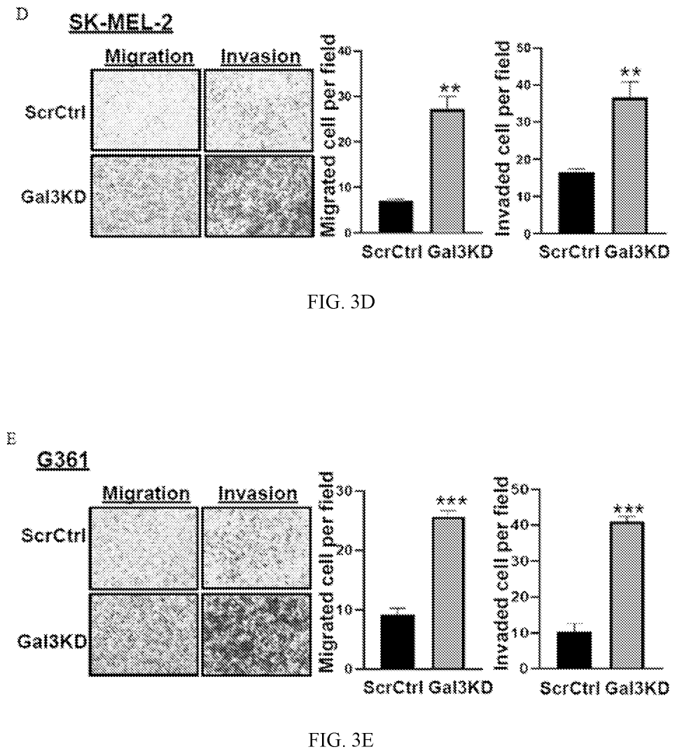

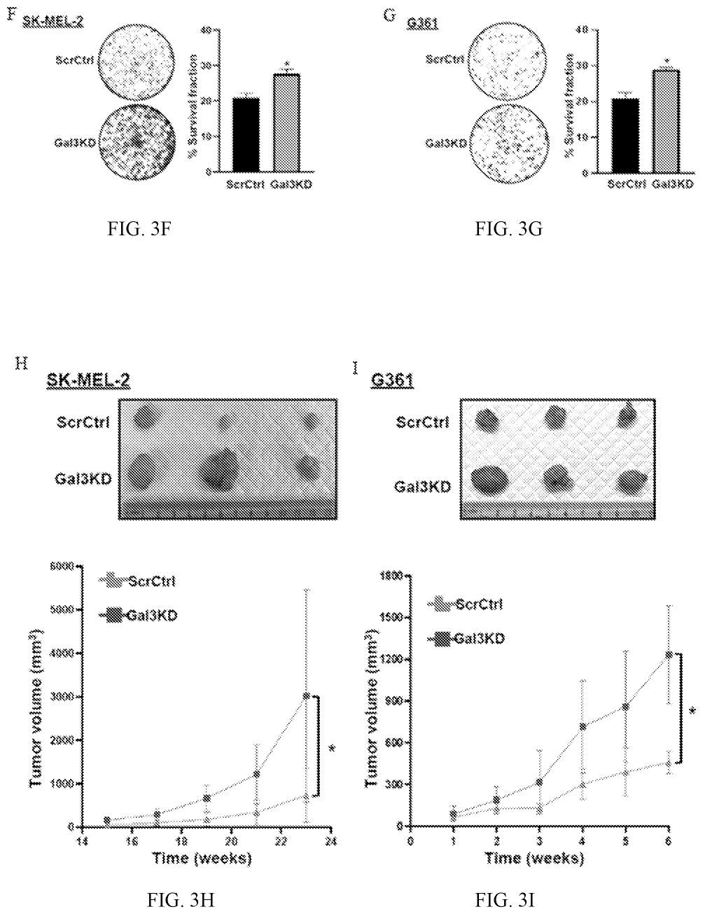

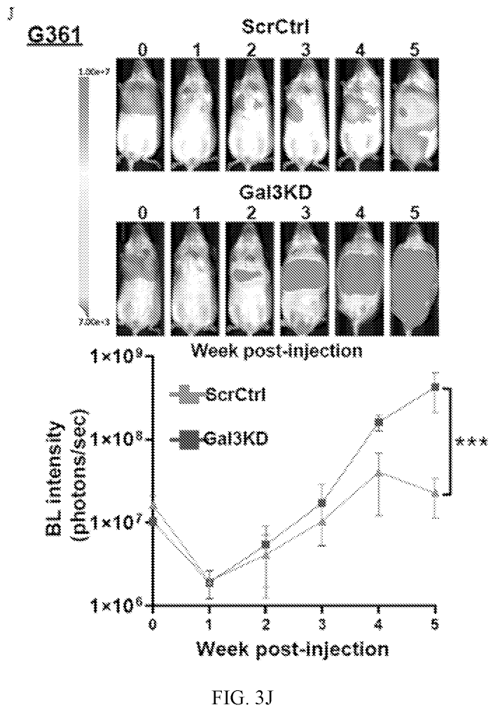

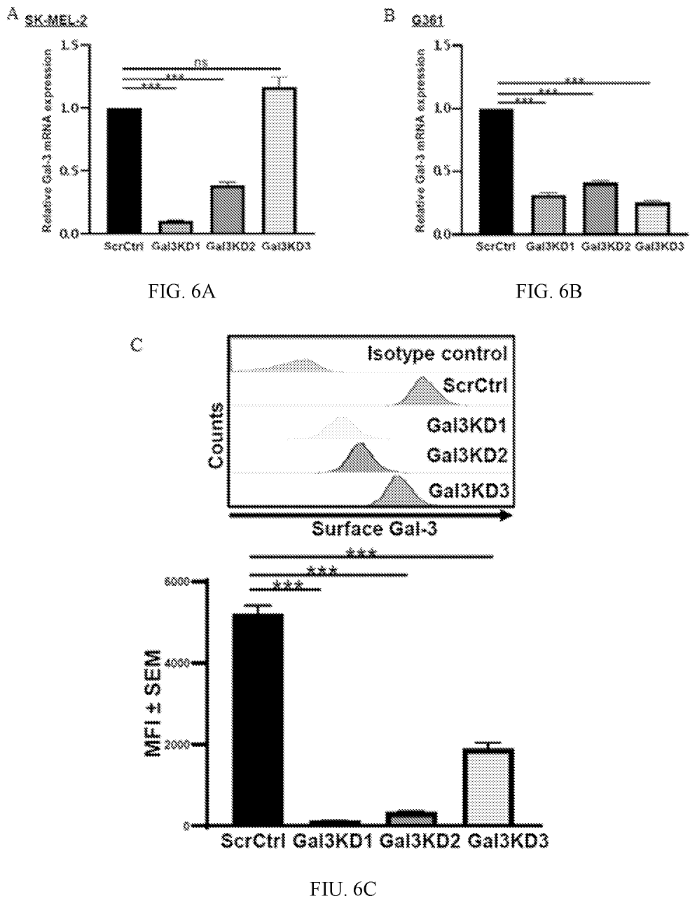

To determine the functional consequences of Gal-3 downregulation in melanoma cells, SK-MEL-2 and G361 cell lines were selected for experimental Gal-3 silencing due to their high inherent Gal-3 expression plus the low metastatic phenotype of SK-MEL-2, making them ideal for testing the hypothesis. First, Gal-3 knockdown lines of SK-MEL-2 and G361 cells were established by lentiviral-mediated transduction of cells with three different LGALS3 shRNAs. Stable KD of Gal-3 was verified by immunoblotting (FIGS. 3A and 3B) and RT-qPCR (FIGS. 6A and 6B). Stable KD of Gal-3 in SK-MEL-2 cells was also verified by flow cytometry analysis (FIG. 6C).

LGALS3 shRNA #1 had the greatest knockdown efficiency on Gal-3 expression among the three used constructs in SK-MEL-2 and G361 cells (p<0.001). Hence, the Gal3KD1 lines were used in the entire downstream analyses (hereafter referred to as Gal3KD). A change in the morphology of Gal-3-silenced cells was observed, which appeared bigger in size with frequent giant multinucleated cells (arrows), compared to scrambled control cells (ScrCtrl) (FIG. 3C).

Metastatic abilities of Gal-3-silenced cells were assessed in vitro using the transwell migration as well as invasion assays. The results showed that Gal-3 knockdown significantly enhanced the migration and invasion capacities of SK-MEL-2 cells (p<0.01 and p<0.01, respectively) (FIG. 3D) and G361 cells (p<0.001 and p<0.001, respectively) (FIG. 3E). Lack of Gal-3 also resulted in significantly increased number and size of SK-MEL-2 and G361 colonies (p<0.05 and p<0.05, respectively) when compared to mock control cells (FIGS. 3F and 3G). Next, a xenograft tumor model in immunodeficient NSG mice was established to assess the influence of Gal-3 silencing on tumor growth in vivo. By following tumor volume over time, it was found that loss of Gal-3 significantly promoted SK-MEL-2 and G361 tumor growth in vivo (p<0.05 and p<0.05, respectively) (FIGS. 3H and 3I).

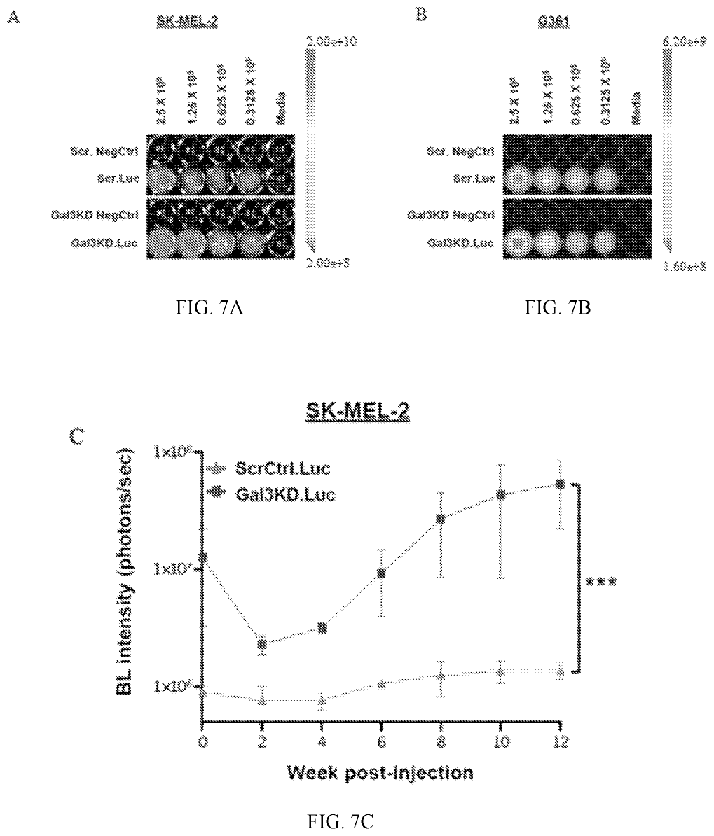

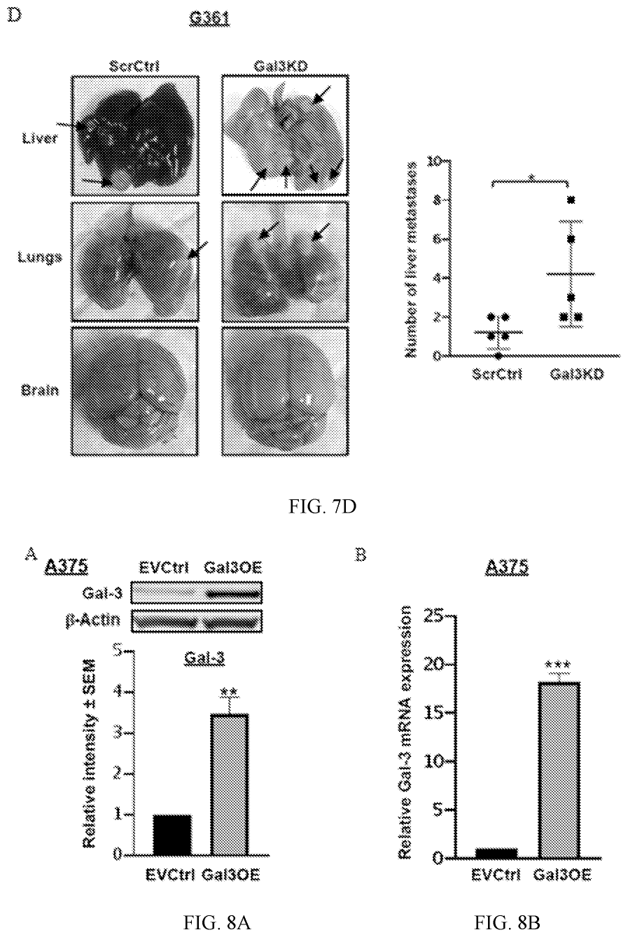

To explore whether Gal-3-depleted cells can enhance metastatic colonization in vivo, SKMEL-2 and G361 cell lines expressing luciferase by lentiviral transduction were first generated. Successful luciferase expression by Gal3KD and ScrCtrl control cells was verified using an in vitro luciferase activity assay (FIGS. 7A and 7B). Then, the luciferase-labeled cells were injected into NSG mice via the tail vein. Bioluminescence imaging was performed weekly using IVIS Spectrum Imaging System until termination of the study. G361 Gal3KD.Luc cells exhibited higher tumor burden compared with Scr.Luc control cells by 5 weeks post-injection (p<0.001) (FIG. 3J), while SK-MEL-2 Gal3KD.Luc cells displayed higher tumor burden compared to mock control cells by 8 weeks post-injection (p<0.001) (FIG. 7C). Furthermore, we screened livers, lungs, and brains from sacrificed mice for macroscopic metastatic colonization. The count of liver metastatic lesions was remarkably augmented in Gal3KD.Luc cells compared to Scr.Luc control cells, while there was no significant difference in number of metastatic lung colonies between the two groups. No metastatic lesions were observed in the brains of either group (FIG. 7D). Altogether, these data indicate that Gal-3 expression is inversely correlated with melanoma progression.

### Example 5—Gal-3 Suppresses Activation of Pro-Oncogenic Signal Pathways and Downregulates the Prometastatic Transcription Factor, NFAT1, in Melanoma Cells

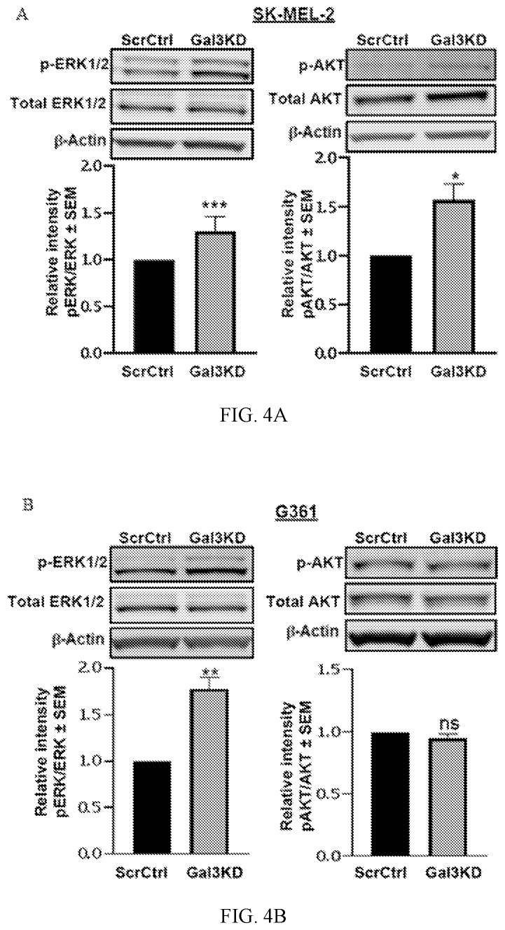

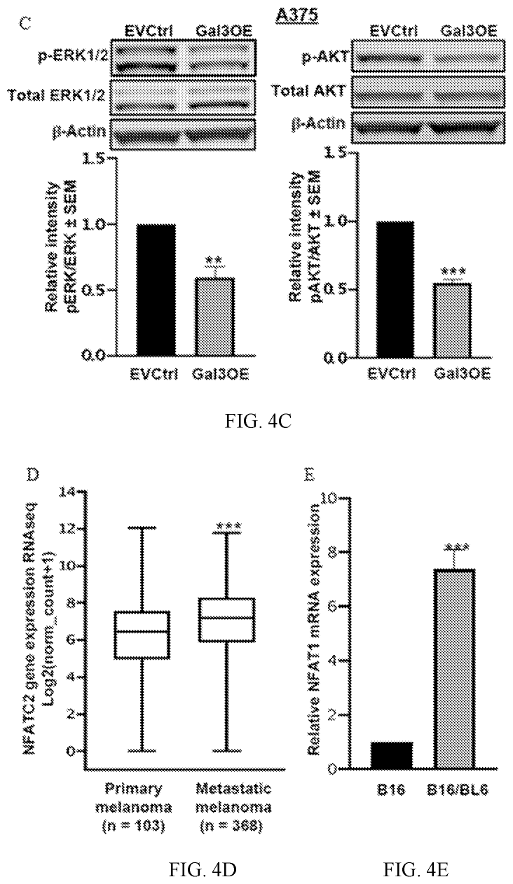

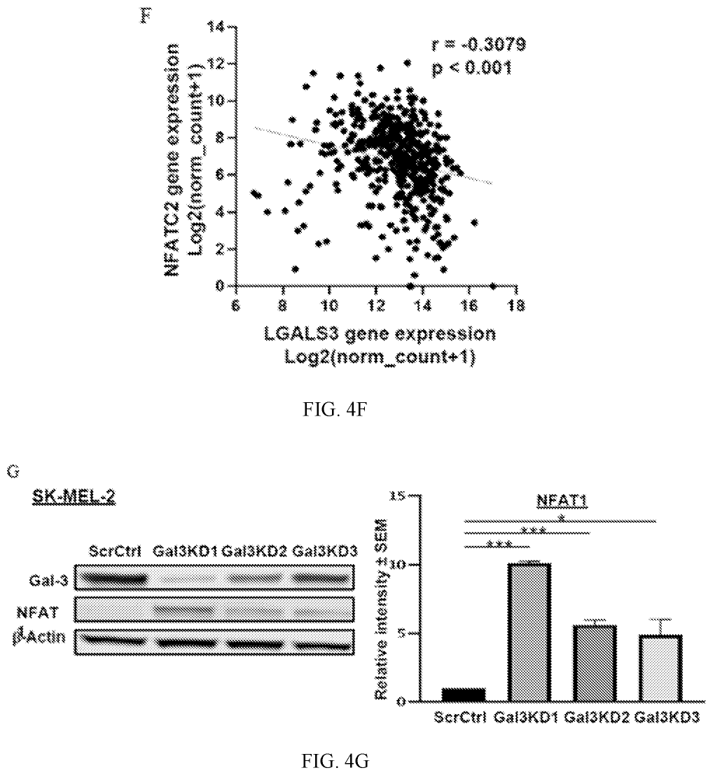

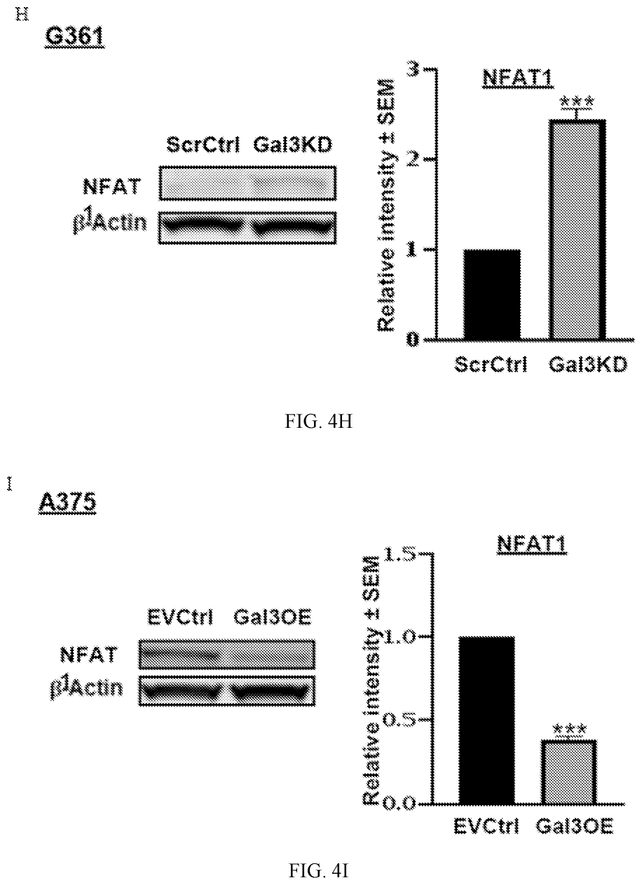

To assess whether Gal-3 silencing triggered canonical intracellular signaling, activation of the pro-survival molecules AKT and ERK1/2 was analyzed. Immunoblotting confirmed the activation of MAPK/ERK and PI3K/AKT signaling pathways in SK-MEL-2 Gal3KD cells versus control cells, as evidenced by increased phosphorylation of ERK1/2 (p<0.001) and AKT (p<0.05) (FIG. 4A), respectively. Similarly, high phosphorylation of ERK1/2 (p<0.01) was detected in G361 Gal3KD cells compared to control cells, while AKT phosphorylation was not affected (FIG. 4B).

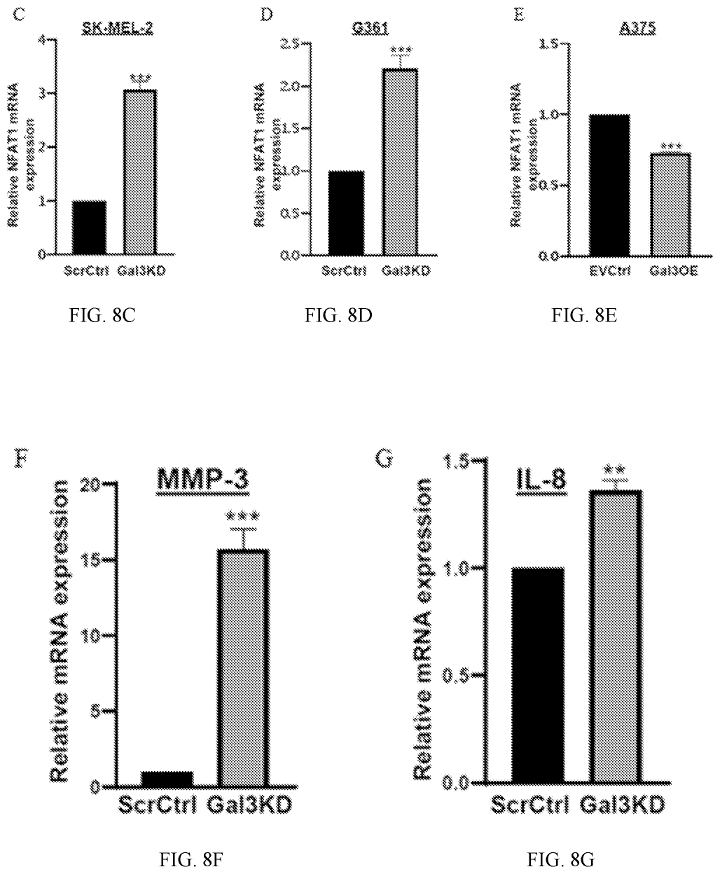

To confirm the suppressive effect of Gal-3 on the oncogenic intracellular signaling pathways, A375 cell line was selected for experimental Gal-3 overexpression due to its low inherent Gal-3 expression. Stable overexpression of Gal-3 was verified by immunoblotting (FIG. 8A) and RT-qPCR (FIG. 8B). The result revealed the suppression of MAPK/ERK and PI3K/AKT signaling pathways upon enforced Gal-3 expression, as evidenced by decreased phosphorylation of ERK1/2 (p<0.01) and AKT (p<0.001) in A375 Gal-3-overexpressing (Gal3OE) cells compared to empty vector control (EVCtrl) cells (FIG. 4C). Together, these data indicate that intracellular Gal-3 regulates the activation of MAPK/ERK and PI3K/AKT signaling pathways in melanoma cells.

NFAT1 is involved in driving melanoma progression. NFAT1 expression was explored in melanoma samples from the TCGA database, and it was found that NFAT1 was upregulated in metastatic melanoma samples compared with primary melanomas (p<0.001) (FIG. 4D). NFAT1 was also upregulated in the highly metastatic B16/BL6 cell line compared with the parental B16 cell line (p<0.001) as evidenced by RT-qPCR analysis (FIG. 4E).

To test the possible relationship between Gal-3 and NFAT1 expression in melanoma, Spearman's correlation analysis was applied to calculate the correlation coefficient between the expression levels of Gal-3 and NFAT1 in the TCGA melanoma database. Data analysis showed that the Gal-3 expression is significantly negatively correlated with NFAT1 expression in melanoma samples (r=−0.3079, P<0.001) (FIG. 4F).

To further investigate the possible negative regulatory role of Gal-3 on NFAT1 expression, NFAT1 expression was assessed in the three established Gal-3-silenced SK-MEL-2 lines (Gal3KD #1, Gal3KD #2, Gal3KD #3) using immunoblotting. Data analysis demonstrated a significant upregulation of NFAT1 in the three lines (p<0.001, p<0.001, and p<0.05, respectively), with an obvious negative correlation with Gal-3 expression levels inside these cells (FIG. 4G). NFAT1 expression was also upregulated in G361 Gal3KD cells versus control cells as evidenced by immunoblotting (p<0.001) (FIG. 4H). On the other hand, NFAT1 expression was downregulated in A375 Gal3OE cells compared to EVCtrl cells as evidenced by immunoblotting (p<0.001) (FIG. 4I). Consistently, RT-qPCR data analysis confirmed NFAT1 upregulation in Gal-3-silenced SK-MEL-2 and G361 cells (p<0.001 and p<0.001 respectively) (FIGS. 8C and 8D) and the downregulation of NFAT1 in Gal-3-overexpressing A375 cells (p<0.001) (FIG. 8E).

NFAT1 was shown to exert its pro-metastatic function in melanoma cells by regulating other effector proteins, such as matrix metalloproteinase (MMP)-3 and interleukin (IL)-8. Therefore, the expression levels of NFAT1 and its target proteins were characterized in Gal-3-depleted melanoma cells using RT-qPCR. Interestingly, the results show a significant upregulation of NFAT1 (p<0.001) (FIG. 8C), MMP-3 (p<0.001) (FIG. 8F), and IL-8 (p<0.01) (FIG. 8G) in SK-MEL-2 Gal3KD cells compared with control cells. Collectively, these results suggest an inhibitory role for Gal-3 on the expression of NFAT1, and hence its downstream effector proteins.

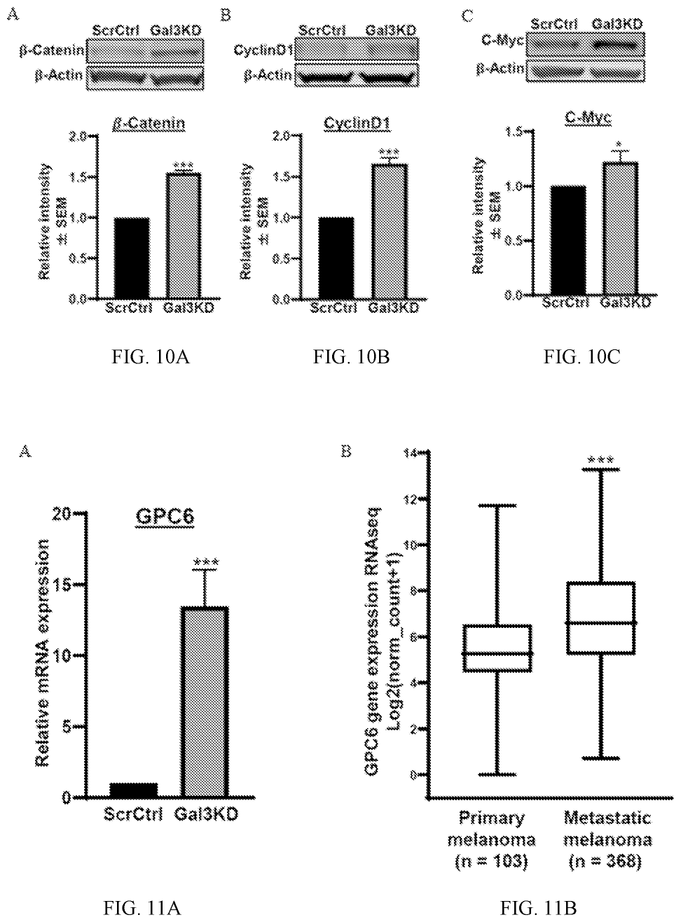

Dysregulation of the Wnt/β-catenin signaling pathway was also implicated in melanoma progression. To test whether Gal-3 downregulation affects Wnt/β-catenin signaling, the protein expression levels of β-catenin, c-Myc, and cyclin D1 were measured, which are important components of the Wnt/β-catenin pathway. The results show that loss of Gal-3 resulted in increased β-catenin (p<0.001) (FIG. 10A), cyclin D1 (p<0.001) (FIG. 10B), and c-Myc expression (p<0.05) (FIG. 10C). Together, these data indicate that intracellular Gal-3 depletion promotes the activation of MAPK/ERK, PI3K/AKT, and Wnt/β-catenin signaling pathways.

### Example 6—Glypican-6 is a Potential Target of Gal-3-NFAT1 Axis in Melanoma

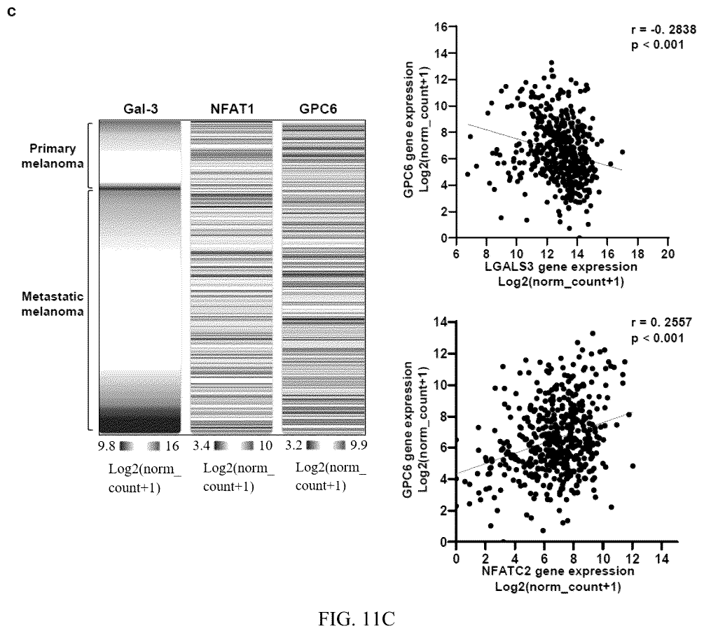

Glypican-6 (GPC6) has been shown to be regulated by NFAT1 in conjunction with its role in mediating tumor cell invasion in breast cancer. Although GPC6 was identified as a potential marker of melanoma metastasis, its regulation by NFAT1 has not been established in melanoma. Here, the result revealed a significant upregulation of GPC6 expression in Gal-3-silenced cells compared to ScrCtrl cells using RT-qPCR (p<0.01) (FIG. 11A). By analyzing TCGA melanoma data, GPC6 was shown to be markedly upregulated in metastatic melanomas compared to primary melanomas (p<0.001) (FIG. 11B).

Moreover, Spearman's correlation analysis was carried out to evaluate the relationships between GPC6 and Gal-3 or NFAT1 in the TCGA melanoma data. Interestingly, a significant negative correlation between GPC6 and Gal-3 expression levels (r=−0.2838, P<0.001) (FIG. 11C, left heatmap and right upper panel) and a significant positive correlation between GPC6 and NFAT1 expression levels (r=0.2557, P<0.001) (FIG. 11C, left heatmap and right lower panel) were observed. These data indicate a negative regulatory role for Gal-3 on GPC6 expression in melanoma cells potentially mediated via NFAT1.

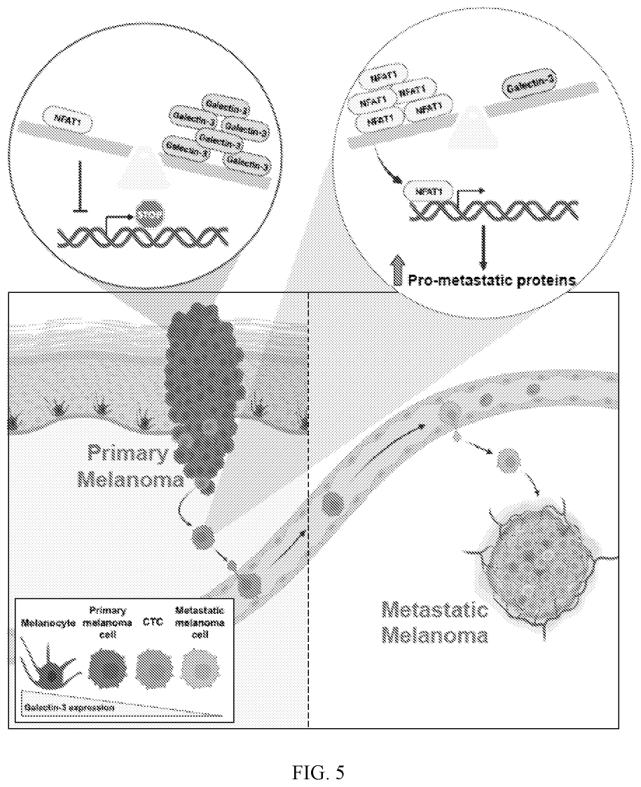

This subject invention is based, in part, in the identification of a negative correlation between Gal-3 expression in melanoma cells and their metastatic potential. A significant trend of Gal-3 loss was observed in metastatic melanoma versus primary melanoma patient cohorts retrieved from TCGA and GEO databases as well as in murine melanoma models. The observations in clinical samples were supported by the in vitro and in vivo studies, which revealed enhanced migration, invasion and metastatic colonization induced by depletion of cellular Gal-3. Furthermore, the invention demonstrated that Gal-3 negatively regulates the expression of the pro-metastatic transcription factor NFAT1 in melanoma cells. Taken together, the results strongly suggest a metastasis-suppressive role of intracellular Gal-3 in melanoma potentially via downregulating NFAT1 (FIG. 5).

The inventors suggest that the release of MMPs from growing melanomas eventually can consume intracellular Gal-3 stores, which, in turn, facilitates melanoma cell shedding from the primary tumor into the circulation. The diverse and sometimes opposing roles played by Gal-3 in cancer progression ultimately depend on its cellular localization, which dictates its structure and, in turn, its biological functions. Accordingly, the interplay between intracellular and microenvironmental Gal-3 probably defines the clinical outcome of melanoma. This is evidenced by the observation of significantly high Gal-3 levels in sera of late-stage metastatic melanoma patients compared to healthy individuals despite the remarkable low levels of melanoma cell intrinsic Gal-3.

Interestingly, the data also showed that loss of Gal-3 was associated with increased number of polyploid/multinucleated giant cancer cells (PGCCs). This phenomenon was described in cancer cells as a response to stressful conditions triggered by either endogenous stimuli, such as hypoxic TME or chemotherapeutic agents and ionizing radiation. Although PGCCs have been associated with suppressed proliferative capacity, it is becoming increasingly evident that their presence is associated with a more metastatic phenotype with poorer clinical outcomes.

NFAT1 is well-known for its roles in T cell development and activation. NFAT1 is a metastasis-promoting molecule in melanoma. Mechanistically, MMP-3 has been identified as a downstream target of NFAT1 in melanoma and is known to promote melanoma cell invasive abilities. IL-8, a neutrophil chemotactic factor, is also regulated in melanoma cells by NFAT1 and is known to attract neutrophils and facilitate CTC extravasation. In the present invention, consistent negative correlation between Gal-3 and NFAT1 was observed by analyzing TCGA melanoma data as well as data from murine B16 melanoma models. These observations were supported by data from in vitro studies using Gal-3-silenced as well as Gal-3-overexpressing human melanoma cells, raising the possibility that NFAT1 is negatively regulated, either directly or indirectly, by Gal-3. In conclusion, the data implicate downregulated Gal-3 expression in promoting melanoma metastasis and suggest NFAT1 as consequential mediator of the metastatic behavior.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

1. A method for reducing melanoma tumor growth in a subject, comprising administering to the subject a pharmaceutical composition comprising 1) a nucleic acid sequence that encodes galectin-3 (Gal-3); and/or 2) a vector comprising a nucleic acid sequence that encodes Gal-3, and measuring melanoma tumor volume over time.

2. The method of claim 1, the administration being local, oral, topical, or parenteral administration.

3. The method of claim 1, the subject being a human.

4. The method of claim 1, the melanoma being stage II, III or IV melanoma.

5. The method of claim 1, the melanoma being metastatic melanoma.

6. The method of claim 1, the melanoma being immune checkpoint inhibitor (ICI) therapy-resistant melanoma.

7. The method of claim 1, the subject having been treated with an ICI therapy.

8. The method of claim 7, the ICI being an antibody to cytotoxic T-lymphocyte antigen 4 (CTLA-4).

9. The method of claim 7, the ICI therapy being anti-PD-1 therapy.

10. (canceled)

11. A method for slowing the growth of melanoma cells, the method comprising contacting the melanoma cells with a composition comprising 1) a nucleic acid sequence that encodes Gal-3; and/or 2) a vector comprising a nucleic acid sequence that encodes Gal-3, and measuring the number and size of colonies formed by the melanoma cells, the melanoma cells being resistant to an ICI.

12. The method of claim 11, the melanoma cells being metastatic melanoma cells.

13. (canceled)

14. The method of claim 11, the ICI being an antibody to CTLA-4.

15. A method for inhibiting, suppressing, or slowing down melanoma metastasis in a subject, the method comprising administering to the subject a pharmaceutical composition comprising 1) a nucleic acid sequence that encodes Gal-3; and/or 2) a vector comprising a nucleic acid sequence that encodes Gal-3,
the method further comprising administering to the subject an inhibitor of Gal-3,
measuring melanoma tumor volume over time,
measuring the phosphorylation level of ERK1/2 and AKT in melanoma cells from the subject, and measuring the expression levels of nuclear factor of activated T cells (NFAT1), matrix metalloproteinase (MMP-3), interleukin (IL-8), β-catenin, c-Myc, cyclin D1, and Glypican-6 (GPC6) in melanoma cells from the subject.

16. The method of claim 15, the subject having been treated with an ICI therapy for melanoma.

17. The method of claim 15, the melanoma being ICI therapy-resistant melanoma.

18. The method of claim 15, the administration being local, oral, topical, or parenteral administration.

19. The method of claim 15, the composition being administered prior to, simultaneously with or, after the administration of the ICI therapy.

20. (canceled)

21. The method of claim 15, the inhibitor being GB1211.

22. (canceled)

23. The method of claim 1, further comprising administering to the subject GB1211.

24. The method of claim 11, the composition further comprising GB1211.
